SEQUENCE LISTING (33 sequences)

SEQ ID NO: 1 - DNA, 260 bp, Homo sapiens
  Unsure (159)..(159): n= A, C,G or T
gccgcgcacaccaccatcacgtatccttggttcctgtgggcctcattgcagaggcgatgtggaccggggacacgttcaccagggagattgagcctcagaaggcttggttcctgcccagggatccccagtgggcaggtggccgtccctaggttgggaggnggtttgtccacaggcacctgctctgtgctgctgcgatggggtctgcggtgtcctggcctgcaccgttggccttaacgggctagcatggctgtcatgtgtgt

SEQ ID NO: 2 - DNA, 218 bp, Mus musculus
gccgcccacaccaccatcacgtatccttgtcttcaaatggcccagccctcggcagcatatcctggggggtcagtgggtacatggggtcttccgggagtgtcgctgtgcttaggcgagagggggccacaagaactgtcactgtagtggggggagggagggtcttcatgttcttgcctcaactgttggccttaacaggccagcatggctgtcatgtgtgt

SEQ ID NO: 3 - DNA, 226 bp, Rattus norvegicus
gcggcccacaccaccatcacgtatccttgtctccacgtggccctcgggagcacatcctggggggtcggctcctggggggtcggcgggtacacgagtcttccggaagagttgccgtgcttagacgagagggggtcgagagacctgtcactgcagggggggagggggggtcgtcgtgttcttgcctcaactgttggccttaacaggccagcatggctgtcatgtgtgt

SEQ ID NO: 4 - protein, 53 aa, Homo sapiens
CATCHFYRPPRCSHCSVCDNCVEDFDHHCPWVNNCIGRRNYRYFFLFLLSLSA

SEQ ID NO: 5 - protein, 53 aa, Homo sapiens
CATCRFYRPPRCSHCSVCDNCVEEFDHHCPWVNNCIGRRNYRYFFLFLLSLTA

SEQ ID NO: 6 - protein, 53 aa, Homo sapiens
CPKCCSIKPDRAHHCSVCKRCIRKMDHHCPWVNNCVGENNQKYFVLFTMYIAL

SEQ ID NO: 7 - protein, 53 aa, Homo sapiens
CPKCCCIKPERAHHCSICKRCIRKMDHHCPWVNNCVGEKNQRFFVLFTMYIAL

SEQ ID NO: 8 - protein, 53 aa, Homo sapiens
CKVCQAYKAPRSHHCRKCNRCVMKMDHHCPWINNCCGYQNHASFTLFLLLAPL

SEQ ID NO: 9 - protein, 53 aa, Homo sapiens
CSTCLIRKPVRSKHCGVCNRCIAKFDHHCPWVGNCVGAGNHRYFMGYLFFLLF

SEQ ID NO: 10 - protein, 53 aa, Homo sapiens
CFTCKMFRPPRTSHCSVCDNCVERFDHHCPWVGNCVGRRNYRFFYAFILSLSF

SEQ ID NO: 11 - protein, 53 aa, Homo sapiens
CYTCKIFRPPRASHCSICDNCVERFDHHCPWVGNCVGKRNYRYFYLFILSLSL

SEQ ID NO: 12 - protein, 53 aa, Homo sapiens
CKKCIYPKPARTHHCSICNRCVLKMDHHCPWLNNCVGHYNHRYFFSFCFFMTL

SEQ ID NO: 13 - protein, 53 aa, Homo sapiens
CSTCDLRKPARSKHCSVCNWCVHRFDHHCVWVNNCIGAWNIRYFLIYVLTLTA

SEQ ID NO: 14 - protein, 53 aa, Homo sapiens
CDRCQLIKPDRCHHCSVCDKCILKMDHHCPWVNNCVGFSNYKFFLLFLAYSLL

SEQ ID NO: 15 - protein, 53 aa, Homo sapiens
CHLCKVTVNKKTKHCISCNKCVSGFDHHCKWINNCVGSRNYWFFFSTVASATA

SEQ ID NO: 16 - protein, 53 aa, Homo sapiens
CNLCNVDVSARSKHCSACNKCVCGFDHHCKWLNNCVGERNYRLFLHSVASALL

SEQ ID NO: 17 - protein, 15 aa, Artificial Sequence
  synthetic peptide
KKVSGVGGTTYEISV

SEQ ID NO: 18 - protein, 765 aa, Homo sapiens
MPRSPGTRLKPAKYIPVATAAALLVGSSTLFFVFTCPWLTRAVSPAVPVYNGIIFLFVLANFSMATFMDPGVFPRADEDEDKEDDFRAPLYKNVDVRGIQVRMKWCATCHFYRPPRCSHCSVCDNCVEDFDHHCPWVNNCIGRRNYRYFFLFLLSLSAHMVGVVAFGLVYVLNHAEGLGAAHTTITMAVMCVAGLFFIPVIGLTGFHVVLVTRGRTTNEQVTGKFRGGVNPFTRGCCGNVEHVLCSPLAPRYVVEPPRLPLAVSLKPPFLRPELLDRAAPLKVKLSDNGLKAGLGRSKSKGSLDRLDEKPLDLGPPLPPKIEAGTFSSDLQTPRPGSAESALSVQRTSPPTPAMYKFRPAFPTGPKVPFCGPGEQVPGPDSLTLGDDSIRSLDFVSEPSLDLPDYGPGGLHAAYPPSPPLSASDAFSGALRSLSLKASSRRGGDHVALQPLRSEGGPPTPHRSIFAPHALPNRNGSLSYDSLLNPGSPGGHACPAHPAVGVAGYHSPYLHPGATGDPPRPLPRSFSPVLGPRPREPSPVRYDNLSRTIMASIQERKDREERERLLRSQADSLFGDSGVYDAPSSYSLQQASVLSEGPRGPALRYGSRDDLVAGPGFGGARNPALQTSLSSLSSSVSRAPRTSSSSLQADQASSNAPGPRPSSGSHRSPARQGLPSPPGTPHSPSYAGPKAVAFIHTDLPEPPPSLTVQRDHPQLKTPPSKLNGQSPGLARLGPATGPPGPSASPTRHTLVKKVSGVGGTTYEISV

SEQ ID NO: 19 - DNA, 3520 bp, Homo sapiens
ccgcgggtcctgcgccgcgtccagcccgcccgcccgaccccggcccgaccccggccggccctgcccgcccggccccggggagggatgcggcggcgcggcgcccaggatgccccgcagccccgggacgcgcctcaaacccgccaagtacatcccggtggccacggccgccgcgctgctggtcggctccagcaccctcttcttcgtgttcacgtgcccgtggttgacacgagctgtgtccccagctgttcccgtctacaatggcatcatcttcctctttgtcctggccaacttcagcatggccactttcatggaccctggtgttttcccccgagcggatgaggatgaggacaaggaggacgacttccgggctccgctgtacaagaacgtggatgtgcgaggtatccaggtccgcatgaagtggtgtgccacgtgccacttctaccgcccgccgcgctgctcccactgcagcgtctgtgacaactgtgtagaggactttgaccaccactgcccctgggtcaacaactgcatcgggcgtcgaaactatcgctacttcttcctgttcctgctgtcactcagtgcacacatggtgggcgtcgtggccttcggcctggtctacgtgctgaaccacgctgaggggctgggagccgcgcacaccaccatcaccatggctgtcatgtgtgtggccggcctcttcttcatccctgtcattggcctcactggcttccatgtggtgctggtcactcgggggcgcaccaccaacgagcaggtgactgggaagttccgcgggggtgtgaaccctttcacccgaggctgctgtgggaatgtggagcacgtgctgtgtagccccctggcgccccggtacgtggtggagccaccccggctgccgctcgcggtgagtttgaagccgcctttccttaggcctgaactcctggaccgagctgcaccgctcaaggtcaagcttagtgacaacgggctgaaggctggcctgggccgtagcaagtccaagggcagcctggaccggctggatgagaagccactggacttggggccaccactgccccccaagatagaggctggcacgttcagcagtgacctgcagaccccgcgcccaggcagtgctgagagtgccctgtcggtgcagaggaccagccccccgacacctgccatgtacaagtttaggccggctttccccacgggtcccaaggtgcccttctgtggaccaggcgagcaggttccaggccctgattccctgaccctgggggacgacagcatccgtagcctggactttgtgtccgagccgagcctggacctccctgactatgggccagggggcctgcatgcagcctacccgccatccccaccgctcagcgcctctgatgccttctcgggcgctttgcgctccctgagcctcaaggcctcgagccggcggggcggggatcatgtggccctgcagcccctgcgctctgagggggggccccccacgccccaccgtagcatttttgccccccatgcactgcccaaccgcaacggcagcctgtcctatgacagcctgctcaatcctggctcgcctggtggccacgcctgccctgcccacccagcagttggcgtggccggataccactcaccctacctgcatcctggggcaacgggcgacccgccacggcccctaccccgcagcttcagccccgtgctgggcccccgcccccgggagccctcgcctgtgcgctacgacaacctgtccaggaccatcatggcatccatccaggagcgcaaggacagggaggagcgtgagcgcctgctgcgctcccaggccgactcactcttcggcgactcaggcgtctatgacgctcccagctcctacagcctgcagcaggccagtgtgctgtccgagggcccccgaggtcccgcgctgcgctatggctccagagacgaccttgtggctgggcccggcttcggtggcgcccgcaaccctgccctgcagacgtcactgtcctcgctgtccagctccgtgagccgtgcaccgcggacgtcgtcctcctccctgcaggctgatcaggccagcagcaacgccccggggccccggcccagcagtggctcacacaggtcacctgcacgccagggcctgccctccccgcccggcactccccactcaccatcctacgcgggccccaaagctgtcgccttcatccacacggacctcccagagccaccgccctcgctgaccgtgcagagggaccaccctcagctgaagactcccccaagtaagcttaatgggcagtccccgggcctggcccggctgggacctgccaccggccccccagggccctctgccagccctacacggcacacgctggttaagaaggtgtccggcgtgggtgggaccacctacgagatctcggtgtgaggactgactgccacacatccgccatggtgccacggggaccaggaccccacagcgcaccccccctccccaccaacttctctgccccagggacccgaggccaccccagcctggtgtggacccatcggcgggagagagtgccacgcctccacagcttgccccaagcgctctgcctgcccgtccactcatctgcccatggggaagtcggctcactgggacaagggccactgggctggtctgtgtctgggcctgtcccatggctggggcagtgagggggcccagtcagcctctttggggcaccctctctcagccaggcttggcccactgccatcacccagcaccccagatcaccgccaggccagcccccaatggtccccttacggacaggtcccagagatggacagaggcacccagggcccccaccgtccttctgacacagcctgtgggctcccggaccgagtgtcccccgccaggctactcctaactaacgcgttgcctttcacggaccccgctggaagcttgtagcttggcaaggctgatgcttctgccctggcctgctctgggtggtggtggataggtggacagacggccagccagccagctgtggccgggggcccggctccatgtgtcccgtgtctgtgtgctgtgctgccgcgccgtgtctgatgtgtcagtgctccggccgccgctgtccctttcatcaaagccttaacctttgctttatgctcttgtgggaggcgacgggggggcaggcgggagcaggcacgggggtgatgctgccacagggggctggtgacacccagagccccctccccagccctcaggccctccctgccaaactggagaaccccaccccaaggcatgccacgtccgcagccccggcctggctgcggtgctcgcgccgtgggaaagcacactggggaggggtcagtgcttcccttggtgtcagggacctgagagtaagcacatgacagcgtctgcttgcgttgtgtctgttttatgtttttatatctacatctatatatctataattttattaaaaaaaagaaaaagaaaaaaaaaaaaaaaaaaaaaaaaaaaaaaaaaaaaaaaaaaaaaaaaaaaaaaaaaaaaaaaaaa

SEQ ID NO: 20 - DNA, 32 bp, Artificial Sequence
  synthetic oligonucleotide
cggaattcgccgccaccatgccccgcagcccc

SEQ ID NO: 21 - DNA, 34 bp, Artificial Sequence
  synthetic oligonucleotide
cgggatcctatcacaccgagatctcgtaggtggt

SEQ ID NO: 22 - DNA, 23 bp, Artificial Sequence
  synthetic oligonucleotide
cagccccgggacgcgcctcaaac

SEQ ID NO: 23 - DNA, 24 bp, Artificial Sequence
  synthetic oligonucleotide
tggccccaagtccagtggcttctc

SEQ ID NO: 24 - DNA, 25 bp, Artificial Sequence
  synthetic oligonucleotide
tggtgtgccacgtgccacttctacc

SEQ ID NO: 25 - DNA, 27 bp, Artificial Sequence
  synthetic oligonucleotide
cattcccacagcagcctcgggtgaaag

SEQ ID NO: 26 - DNA, 18 bp, Artificial Sequence
  synthetic oligonucleotide
tggtgtgccacgtgccac

SEQ ID NO: 27 - DNA, 18 bp, Artificial Sequence
  synthetic oligonucleotide
cctggtctacgtgctgaa

SEQ ID NO: 28 - DNA, 18 bp, Artificial Sequence
  synthetic oligonucleotide
ggtttgtccacaggcacc

SEQ ID NO: 29 - DNA, 18 bp, Artificial Sequence
  synthetic oligonucleotide
tagaaggcacagtcgagg

SEQ ID NO: 30 - DNA, 34 bp, Artificial Sequence
  synthetic oligonucleotide
ccggatatctggtgtgccacgtgccacttctacc

SEQ ID NO: 31 - DNA, 44 bp, Artificial Sequence
  synthetic oligonucleotide
aaggaaaaaagcggccgcattcccacagcagcctcgggtgaaag

SEQ ID NO: 32 - DNA, 25 bp, Artificial Sequence
  synthetic oligonucleotide
atcaccatggctgtcatgtgtgtgg

SEQ ID NO: 33 - DNA, 23 bp, Artificial Sequence
  synthetic oligonucleotide
ccaacgagcaggtgactgggaag

(19) United States
(12) Patent Application Publication
(10) Pub. No.: US 2008/0254462 A1
(43) Pub. Date: Oct. 16, 2008

(54) ALTERED ZDHHC8 EXPRESSION AS A MARKER OF INCREASED RISK OF SCHIZOPHRENIA

(76) Inventors: Joseph A. Gogos, Riverdale, NY (US); Maria Karayiorgou, Riverdale, NY (US); Jun Mukai, New York, NY (US)

Correspondence Address: BAKER BOTTS L.L.P., 30 ROCKEFELLER PLAZA, 44TH FLOOR, NEW YORK, NY, 10112-4498, US

(21) Appl. No.: 11/947,361

(22) Filed: Nov. 29, 2007

Related U.S. Application Data
(63) Continuation of application No. PCT/US06/21446, filed on Jun. 1, 2006.
(60) Provisional application No. 60/686,178, filed on Jun. 1, 2005.

(51) Int. Cl. C12Q 1/68

(52) U.S. Cl. 435/6

(57) ABSTRACT

The present invention relates to the role of mutations that perturb expression of ZDHHC8 in increased susceptibility to schizophrenia. It is based, at least in part, on the discovery of a mechanism by which a high risk allele disrupts expression of functional ZDHHC 8, and on the discovery that two proteins involved in synaptic pathways associated with schizophrenia interact with ZDHHC8, at least one of which is a substrate for that enzyme.

### PRIORITY CLAIM

This application claims priority to U.S. Provisional Patent Application Ser. No. 60/686,178, filed Jun. 1, 2005, the contents of which is hereby incorporated by reference in its entirety herein.

### GRANT INFORMATION

The subject matter of this application was developed, at least in part, under a grant from the United States National Institute of Mental Health Grant No. RO1 MH067068, so that the United States Government holds certain rights herein.

### 1. INTRODUCTION

The present invention relates to the role of mutations that perturb expression of ZDHHC8 in increased susceptibility to schizophrenia. It is based, at least in part, on the discovery of a mechanism by which a high risk allele disrupts expression of functional ZDHHC8, and on the discovery that two proteins involved in synaptic pathways associated with schizophrenia interact with ZDHHC8, at least one of which is a substrate for that enzyme.

### 2. BACKGROUND OF THE INVENTION

Microdeletions in chromosome region 22q11 are associated, with relatively high frequency, with severe mental illness. Between one out of three and one out of four subjects with a microdeletion in 22q11 develop schizophrenia or schizoaffective disorder (1-4, 13). A background 0.025% occurrence rate of these deletions in the general population (5, 13) increases to a rate of up to 2% of adult schizophrenic patients (6, 13) and up to 6% of cases with childhood-onset schizophrenia (7, 13). Accordingly, whereas a person in the general population has a risk of about 1% of developing schizophrenia, that risk may be 25-30 times higher in persons having a deletion in the 22q11 region (13). Additional studies have reported suggestive linkage results in the 22q11 region (8, 9, 13) and that schizophrenic patients carrying a 22q11 deletion bear the hallmark neuropsychological and neuroanatomical features of classical schizophrenia (10-12, 13). It therefore seems likely that the 22q11 region harbors genes that, alone or in combination, are causally implicated in schizophrenia in a subset of patients.

Although the majority (87%) of 22q11 deletions are 3 Mb in size, a “schizophrenia critical region” confined to 1.5 Mb has been identified; as the deletions are mediated by low copy repeat (“LCR”) sequences, this critical region has been denoted “LCR-A to -B” (6, 13, 14, 15). The majority of the genes in the region are known (http://genome.ucsc.edu), making this locus amenable to a molecular genetic analysis, and Liu et al. (16) performed linkage disequilibrium (LD) studies in family samples to evaluate whether nondeletion common variants of individual genes within the 22q11 region might play an even greater role than microdeletions in susceptibility to schizophrenia in the general population (13). The LD analysis delineated a subregion of 22q11 in which PRODH2 and DGCR6 are the only known genes, and identified a relatively common haplotype where the schizophrenia susceptibility variant(s) likely reside (16, 13). While the conclusions of these studies implicated PRODH2 as playing an important role, comparison of the increase in the morbid risk of schizophrenia associated with the identified PRODH2 variation to the risk associated with the 22q11 microdeletion could not exclude contribution from other genes in the region (16, 17, 13). Further LD studies (13) revealed a limited number of candidate genes, including two membrane-associated proteins, KIAA1292 (now known as ZDHHC8) and NOGO-R, which are highly expressed in brain regions implicated in schizophrenia.

### 3. SUMMARY OF THE INVENTION

The present invention relates to the role of mutations that perturb expression of ZDHHC8 in increased susceptibility to schizophrenia and related disorders. It is based, at least in part, on the discoveries that (i) a point mutation associated with increased risk of schizophrenia prevented proper splicing of ZDHHC8 mRNA; (ii) ZDHHC8 interacts with two homologous proteins (PSD95 and PSD93) which are key signaling molecules in glutamatergic synapses; (iii) ZDHHC8 palmitoylates and facilitates membrane translocation of PSD95; and (iv) palmitoylation of PSD95 by ZDHHC8 is necessary for maintaining a homeostatic balance between excitatory glutamatergic and inhibitory GABAergic synapses.

In a first set of embodiments, the present invention provides for methods of identifying a subject at risk of developing schizophrenia or a related disorder, comprising identifying, in the individual, a mutation that decreases or prevents expression of wild-type ZDHHC8 when compared to a subject not diagnosed with schizophrenia or a related disorder. In related embodiments, the invention may be used to aid in the diagnosis of schizophrenia or a related disorder in a subject.

In a second set of embodiments, the present invention provides for a transgenic non-human animal in which expression of at least one copy of endogenous ZDHHC8 is decreased or prevented. In preferred embodiments, expression of both copies of endogenous ZDHHC8 is decreased or prevented, and most preferably, detectable expression of the active protein is absent.

In a third set of embodiments, the present invention provides for methods and assay systems for identifying agents that may be used to treat schizophrenia or a related disorder in a subject carrying a mutation that decreases or prevents ZDHHC8 expression. As one non-limiting example, a test agent may be administered to a transgenic animal in which expression of one or both copies of ZDHHC8 is decreased or prevented, and the animal may be tested for behavior, such as spontaneous exploratory behavior, where an increase in such behavior bears a positive correlation with the ability of the test agent to treat schizophrenia or a related disorder. In another non-limiting example, a cell in which endogenous ZDHHC8 expression is decreased or prevented may be exposed to a test agent, and the level of PSD93 or PSD95 activity, palmitoylation, and/or membrane translocation may be measured, where an increase in the activity, palmitoylation, and/or membrane translocation of either PSD93 or PSD95, especially PSD95, bears a positive correlations with the ability of the test agent to treat schizophrenia or a related disorder. In another non-limiting example, a cell in which endogenous ZDHHC8 expression is decreased or prevented may be exposed to a test agent, and the ratio of excitatory glutamatergic to inhibitory GABAergic synapses may be measured, where an increase in the ratio of excitatory glutamatergic to inhibitory GABAergic synapses bears a positive correlations with the ability of the test agent to treat schizophrenia or a related disorder.

In a fourth set of embodiments, the present invention provides for a method of treating a subject suffering from schizophrenia or a related disorder comprising administering, to the subject, an effective amount of an isolated nucleic acid encoding human ZDHHC8, operably linked to a suitable promoter, optionally comprised in a suitable vector molecule.

### 4. BRIEF DESCRIPTION OF THE FIGURES

FIG. 1A-D. Genomic analysis of the ZDHHC8 locus. (A) Associated subregion at the distal part of the 22q11 locus. Arrows and arrowheads indicate the SNPs tested from within the 80 kb haplotypic block (13). Arrows indicate the 5 SNPs showing significant association with schizophrenia in our US and Afrikaner family samples (13). The position of the most significantly associated SNP, rs175174, within intron 4 of the ZDHHC8 candidate gene is indicated. Vertical bars represent the approximate distribution of exons of genes residing within or immediately adjacent to the 80 kb-haplotypic block. (B) Genomic organization of ZDHHC8. The exon/intron structure of the ZDHHC8 gene is shown along with the position of SNP rs175174 in intron 4. Genomic sequence of ZDHHC8 was drawn from the April 2002 freeze of Santa Cruz database (http://genome.ucsc.edu). Confidence values for the 5′ splice sites were calculated by using NetGene2 worldwide web server (http://www.cbs.dtu.dk). (C) Identification of alternatively spliced forms of ZDHHC8 and analysis of the predicted proteins. Total RNA was purified from human lymphocytes and reverse-transcribed using a random hexamer. The transcript of ZDHHC8 was then amplified by using primer pair F1-ZDHHC8/R1-ZDHHC8 (TABLE 3) from exons 1 and 8 respectively, the PCR products were separated on a 1.2% agarose gel, purified and sequenced. Fragment B, which had given mixed signals in sequencing, was subcloned and plasmids from 20 isolated single colonies were purified and sequenced. The DNA sequences of the alternatively spliced forms of ZDHHC8 were edited and translated to amino acids by using sequence software Editseq (DNA Star Inc.). The predicted protein sequences were then analyzed by using TMHMM prediction server (http://www.cbs.dtu.dk) for prediction of transmembrane helices and CD-Search (http://www.ncbi.nlm.nih.gov/Structure/cdd) for identification of the ZDHHC domain. (D) Sequence and predicted secondary structure of ZDHHC8 intron 4. A sequence alignment of ZDHHC8 intron 4 from Homo sapiens (SEQ ID NO.: 1), mouse (SEQ ID NO: 2) and rat (SEQ ID NO: 3) is shown indicating (shaded) blocks of conserved sequence identity. The suboptimal 5′ splice site is indicated by two dots; the position of rs175174 is indicated by an arrowhead; the position of the in-frame premature STOP codon is also indicated by asterisks.

FIG. 2A-C. SNP rs175174, modulates the levels of the ZDHCC8 form that retains intron 4. (A) Semi-quantitative polyacrylamide gel electrophoresis of the minigene products. Total RNA was isolated from HEK 293 cells transfected with the ZDHHC8 minigene and was reverse-transcribed using Primer R-BGHpA (TABLE 3) in order to distinguish the minigene from endogenous ZDHHC8 gene. The cDNA was then amplified by using primer pair F1-ZDHHC8/R1-ZDHHC8 (TABLE 3) from exons 3 and 6 respectively. Histograms represent the ratio of the unspliced to the fully spliced form (see Methods) (L=Lymphocytes). (B,C) Quantification of ZDHHC8 intron 4 retention using real-time quantitative (Taqman) PCR assay. The design of the experiment is outlined. Histograms represent the ratio of the unspliced to the fully spliced form. Primer pair F4-ZDHHC8/R-BGHpA and Probe1-ZDHHC8 (TABLE 3) were used to quantify amplicon intron 4(−); primer pair F5-ZDHHC8/R-BGHpA and Probe2-ZDHHC8 (TABLE 3) were used to quantify amplicon intron 4(+). The relative level of the expression was calculated from the ratio of the intron 4(+) cycle threshold (Ct) to intron 4(−) Ct values, both of which were normalized to control transcript GAPDH Ct value using the formula 2x[ΔCt (intron 4(+)−ΔCt (intron 4(−)], while ΔCt representing the difference in Ct values between the minigene transcript and the control transcript. A similar protocol was used for real-time quantitative RT-PCR on total fetal brain RNA (c) except that a primer from exon 8 was used.

FIG. 3A-C. Structure and subcellular localization of ZDHHC8 gene product. (A) The ZDHHC family of putative palmitoyltransferases at Chromosome 22 (SEQ. ID. NO.: 4), Chromosome 11 (SEQ. ID. NO.: 5), Chromosome 3 (SEQ. ID. NO. 6), Chromosome 16B (SEQ. ID. NO. 7), Chromosome 10B (SEQ. ID. NO.: 8), Chromosome 12 (SEQ. ID. NO. 9), Chromosome 1 (SEQ. ID. NO. 10), Chromosome X (SEQ. ID. NO.: 11), Chromosome 10A (SEQ. ID. NO.: 12), Chromosome 7 (SEQ. ID. NO. 13), Chromosome 8 (SEQ. ID. NO.: 14), Chromosome 5 (SEQ. ID. NO. 15) and Chromosome 16A (SEQ. ID. NO. 16). The predicted ZDHHC8 domain structure is shown for the transmembrane domains (I-IV), ZDHHC domain and a putative C-terminal PDZ domain. Also shown is a sequence alignment of ZDHHC motifs from Homo sapiens indicating (underlined) critical residues of the ZDHHC, including a DHHC motif consisting of the amino acids Asp-His-His-Cys (DHHC) and a cysteine-rich domain (zf) that includes a Cys4 zinc-finger-like metal binding site. (B) Sub-cellular localization of ZDHHC8. Representative HeLa cells transfected with ZDHHC8 and analyzed using confocal microscopy. ZDHHC8 was visualized using 280GP antibody followed by incubation with a Cy3-labeled secondary antibody (striped arrowhead). Expression of the cis-Golgi marker GM130, trans-Golgi marker golgin-97, late endosomal marker mannose 6-phosphate receptor (M6PR) and early endosomal marker EEA1 were detected with an Alexa 488-conjugated secondary antibody (arrow). Nucleus was visualized using To-Pro-3 iodide (arrowhead). Images were merged electronically (striped arrow). Identical localization patterns were obtained with two separate ZDHHC8 forms with presumably impaired palmitoyltransferase activity: a form with a C134A mutation introduced into the DHHC motif (DHHA) of the ZDHHC domain—which likely constitutes a core element of a palmitoyltransferase activity domain (27), as well as an alternative splicing form, which lacks a DHHC-CRD domain suggesting that this sub-cellular distribution pattern is independent of the enzymatic activity. (C) The distribution of ZDHHC8 in cultured hippocampal neurons. Neurons (DIV9) co-transfected with ZDHHC8 and YFP-PSD-95 were stained with 280 GP antibody followed by incubation with a Cy3-labeled secondary antibody (arrowhead). YFP-PSD-95 expression was visualized at low magnification (i) and high magnification (iii) by direct fluorescence (arrow). Endogenous NMDAR2B was stained with NMDAR2B antibody (ii) followed by incubation with a Cy5-labeled secondary antibody (striped arrowhead). In all cases, no staining was observed in control sections when primary antibodies were omitted.

FIG. 4A-D. Targeted inactivation of the mouse Zdhhc8 locus. (A) Brain expression of the mouse Zdhhc8. In situ hybridization analysis on adult mouse brain using a probe from the 3′-UTR of the mouse Zdhhc8 gene (arrowhead). (B) Targeting of mouse Zdhhc8 locus in embryonic stem cells and mice. For the construction of the targeting construct, a Zdhhc8 genomic fragment, encompassing exons 2-11 was replaced by the self-excisable pACN cassette including the neo gene selectable marker (41). Cell culture, electroporation and generation of the chimeric mice were performed essentially as previously described. About 5% of the tested embryonic stem cell clones were positive for homologous recombination and two clones were selected for injection into C57B6 blastocysts. Chimeric males were mated with C57BL/6 females and DNA from tail biopsies of F1 agouti coat pups was typed by Southern blotting and PCR at the Zdhhc8 genomic locus. F1 heterozygous mice were mated, and F2 mice of all three genotypes were obtained. The probe used for Southern analysis is shown as a thick black line. The diagnostic BamH1 restriction fragments (7.5 or 8.5 kb) are underlined. Genomic Southern blot analysis of tail biopsies as well as brain RT-PCR are shown in the lower right. Genomic DNA was isolated from offspring obtained after breeding of heterozygous mice, digested with BamH1, and probed with a 1-kb fragment adjacent to the right arm of the targeting construct: wild-type and recombinant restriction fragments are 14 kb and 8.5 kb, respectively. (C) Gross brain morphology. Representative photomicrographs showing Nissl staining from adult female wild-type (WT; left) and homozygous Zdhhc8 mutant mice (Hom; right) in frontal cortex (−1.94 mm from Bregma, top), dorsal hippocampus (approx. −1.46 mm from Bregman, middle) and auditory cortex (−2.92 mm from Bregman, bottom). (D) Sensorimotor gating. Prepulse inhibition assay using combinations of one startle level (120 dB) and two prepulse levels (78 dB and 82 dB) in homozygous (Hom) and heterozygous (Het) Zdhhc8 mutant female mice and wild-type (WT) littermate controls. Higher Y-axis values represent greater percent inhibition. No significant decreases in startle response amplitudes were identified among genotypes. Statistical analysis was performed using ANOVA repeated measures. *P<0.05 versus wild-type control.

FIG. 5A-F. Targeting of the mouse Zdhhc8 locus affects exploratory activity. (A-D) Locomotor response to novelty. (A-C) Three measures of exploratory activity in response to novelty are shown in homozygous and heterozygous Zdhhc8 mutant female mice and wild-type littermate control mice. (A) The total distance traveled (B), the number of rearings (vertical moves) (C) and the number of entries into the centerfield. (D) Total distance traveled is shown for homozygous and heterozygous Zdhhc8 mutant male mice and wild-type littermate control mice. Both total counts (bar graphs) and time course of the effect of the mutation over 5-minute intervals (line graphs) are shown for each measurement. Het, heterozygous; Hom, homozygous; WT, wild-type. (E, F) Locomotor activating effects of MK801. Locomotor stimulation after MK801 injection was observed in both genotypes in both females (E) and males (F). Homozygous Zdhhc8 mutant females were less sensitive to MK801 in terms of the ratio of activity before injection to activity after injection. Time course of the locomotor activity for 30 minutes before and 30 minutes after injection of MK801 or vehicle is shown over 5-min intervals for each genotype. Bar graphs show the pre- to post-injection total activity ratios. Arrowheads indicate the time of MK801 injection. (A-F)* P<0.05, ** P<0.01 and *** P<0.001 versus wild-type controls.

FIG. 6A-C. A. Yeast two-hybrid analysis showing association between the ZDHHC8 C-terminus and PSD93 and PSD95. B. Co-immunoprecipitation of ZDHHC8 and PSD95. C. Co-immunoprecipitation of ZDHHC8 and PSD93. 280GP is an antibody that was generated by immunizing guinea pigs with a synthetic peptide derived from the C-terminal sequence of human ZDHHC8 (KKVSGVGGTTYEISV) (SEQ. ID. NO.: 17).

FIG. 7A-B. ZDHHC8 palmitoylates PSD95 in a manner that depends on an active catalytic domain and an intact C-terminus. A. Metabolic labeling experiment where the substrate (PSD-95) is co-transfected with increasing amounts of the enzyme (ZDHHC8) in HEK293 cells in the presence of radioactive palmitate (palmitate tagged with tritium). Increasing amount of ZDHHC8 transfers increasing amount of radioactive palmitate to PSD-95 (top panel, lanes 1-5). The middle panel is a loading control for PSD-95 protein level (equivalent amounts present in all lanes); the lower panel is a loading control for ZDHHC8 protein level (showing increasing amount of ZDHHC8 protein from left to right i.e. lanes 1-5, 6-10 and 11-15). The experiment was performed with: a full length (FL) fully functional ZDHHC8 enzyme; a natural variant generated by alternative splicing (deltaDHHC) missing almost the entire catalytic domain; and a mutant designated C134A carrying a substitution in a critical amino acid of the catalytic domain (cysteine to alanine mutation). Both deltaDHHC and C134A demonstrate no activity as revealed by lack of incorporation of radioactive palmitate (lanes 6-15). B. metabolic labeling assay with mutant ZDHHC8 protein that lacks the entire C-terminal domain (mutant “230”), essential for the binding of the substrate. As expected, this form shows no essentially no activity when compared with vector-only control.

FIG. 8. Immunocytochemistry comparing the ability of wildtype ZDHCC8 with mutants ΔDHHC, C134A and 230 to facilitate membrane translocation of PSD95 labeled with YFP. For wild-type ZDHHC8 only, impaired translocation of PSD95 lacking an intact palmitoylation site (YFP-PSD-95 C3, 5S) is also shown.

FIG. 9. Cellular fractionation comparing the ability of wildtype ZDHCC8 with mutants ΔDHHC, C134A (DHHA), and 230 to facilitate membrane translocation of PSD95 labeled with YFP. For wild-type ZDHHC8 only, impaired translocation of PSD95 lacking an intact palmitoylation site (C3, 5S) is also shown. Transfer of the substrate (PSD-95) from the cytoplasmic fraction solubilized in Triton-X (T) to the membrane fraction solubilized by SDS (S).

FIG. 10. Amino acid sequence of human ZDHHC8 (SEQ ID NO: 18).

FIG. 11. Nucleic acid sequence of human ZDHHC8 (SEQ ID NO: 19).

FIG. 12A-C. Zdhhc8 deficiency influences the ratio of excitatory-to-inhibitory presynaptic contacts and reduces dendritic complexity. A. Number of excitatory synapses. Hippocampal neurons were stained at DIV12 from wild type (WT) ZDHHC8 deficient mice for the vesicular glutamate transporter (vGLUT)-1 (arrowhead), a marker for excitatory synapses, and synaptophysin (striped arrowhead), a general marker for presynaptic contacts. vGLUT puncta number was significantly reduced in both heterozygous (Het) and homozygous (KO) neurons although the total presynaptic contacts number was not significantly altered among wild-type (WT), Het, and KO. B. Number of inhibitory synapses. Neurons at DIV12 were stained for the vesicular γ-aminobutyric acid (GABA) transporter (vGAT) (arrowhead), a marker of inhibitory synapses. vGAT puncta number was significantly increased in KO mice. C. Hippocampal neurons at DIV8 were transfected with green fluorescent protein (GFP) as a marker to visualize neuronal morphology. At 24 hours after transfection, cells were fixed and stained with a MAP2 antibody to identify dendrites. Representative confocal images of GFP-expressing neurons are shown, obtained using the LSM510Meta with a Zeiss 63× objective. Each image represents a Z-series projection taken at depth intervals of 0.50 μm. Deficiency of Zdhhc8 causes a decrease in the number of total branch points, total dendritic length, and the number of primary dendrites.

### 5. DETAILED DESCRIPTION OF THE INVENTION

For clarity, and not by way of limitation, the detailed description of the invention is divided into the following subsections:

(i) diagnostic methods based on ZDHHC8;

(ii) transgenic animals;

(iii) assay systems; and

(iv) methods of treatment.

### 5.1 Diagnostic Methods Based on ZDHHC8

The present invention provides for methods for identifying a subject at risk of developing, or suffering from, schizophrenia or a related disorder, comprising identifying, in the individual, a mutation that decreases or prevents expression of wild-type ZDHHC8.

“Schizophrenia” is defined herein as set forth in the Diagnostic and Statistical Manual of Mental Disorders (“DSM-IV”; reference (4)), set forth herein as TABLE 1 (below). Related disorders include schizoaffective disorder, schizophreniform disorder, schizotypal disorder, and schizoid disorder, all as defined in the DSMIV, which is incorporated by reference herein. “Related disorders” also encompass non-human disorders which are considered, in the veterinary art, to be analogous to the above-listed human disorders, and include any one or more of the following in a non-human subject: learning problem, hypoactivity, repetitive behavior, and/or anxious, fearful, or dependent personality.

TABLE 1
Diagnostic Criteria of Schizophrenia

Characteristic symptoms: Two (or more) of the following, each
present for a significant portion of time during a 1-month period (or
less if successfully treated):
delusions
hallucinations
disorganized speech (e.g., frequent derailment or incoherence)
grossly disorganized or catatonic behavior
negative symptoms, i.e., affective flattening, alogia, or
avolition
Note: Only one Criterion A symptom is required if delusions are
bizarre or hallucinations consist of a voice keeping up a running
commentary on the person's behavior or thoughts, or two or more
voices conversing with each other.
Social/occupational dysfunction: For a significant portion of
the time since the onset of the disturbance, one or more major areas of
functioning such as work, interpersonal relations, or self-care are
markedly below the level achieved prior to the onset (or when the
onset is in childhood or adolescence, failure to achieve expected level
of interpersonal, academic, or occupational achievement).
Duration: Continuous signs of the disturbance persist for at
least 6 months. This 6-month period must include at least 1 month of
symptoms (or less if successfully treated) that meet Criterion A (i.e.,
active-phase symptoms) and may include periods of prodromal or
residual symptoms. During these prodromal or residual periods, the
signs of the disturbance may be manifested by only negative symptoms
or two or more symptoms listed in Criterion A present in an attenuated
form (e.g., odd beliefs, unusual perceptual experiences).
Schizoaffective and Mood Disorder exclusion: Schizoaffective
Disorder and Mood Disorder With Psychotic Features have been ruled
out because either (1) no Major Depressive, Manic, or Mixed Episodes
have occurred concurrently with the active-phase symptoms; or (2) if
mood episodes have occurred during active-phase symptoms, their
total duration has been brief relative to the duration of the active and
residual periods.
Substance/general medical condition exclusion: The disturbance is not
due to the direct physiological effects of a substance (e.g., a drug of
abuse, a medication) or a general medical condition.
Relationship to a Pervasive Developmental Disorder: If there is
a history of Autistic Disorder or another Pervasive Developmental
Disorder, the additional diagnosis of Schizophrenia is made only if
prominent delusions or hallucinations are also present for at least a
month (or less if successfully treated).

A subject may be a human or non-human subject, such as a dog, cat, or horse. Preferably, the subject is a human, and may be an adult, infant, child, or fetus.

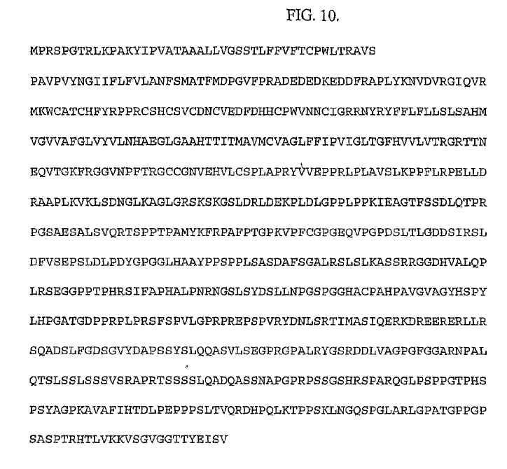

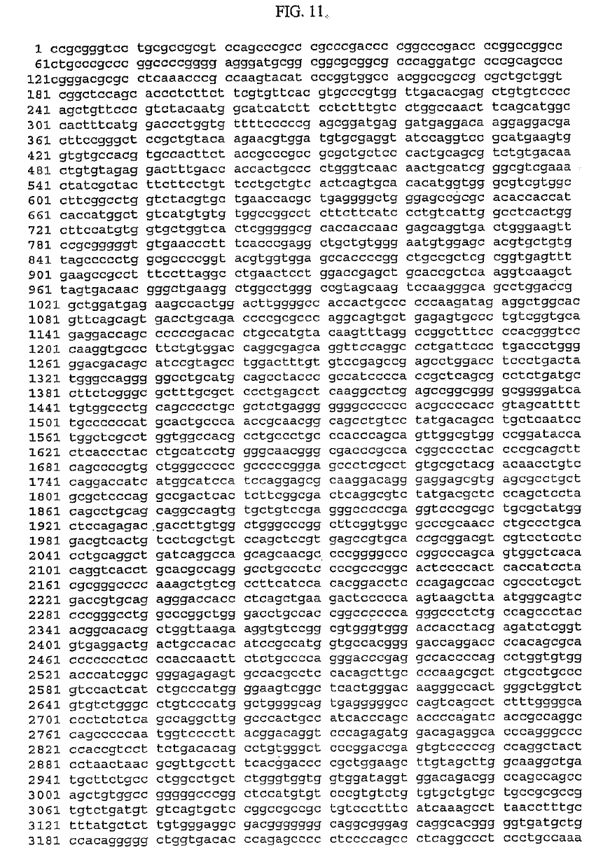

“A mutation that decreases or prevents expression of wild-type ZDHHC8” means a mutation which may be a deletion, substitution, insertion, or inversion, which leads to a decreased level of ZDHHC8 mRNA and/or ZDHHC8 protein and/or ZDHHC8 activity (e.g., palmitoyltransferase activity) in the patient. The wild-type nucleic acid sequence of ZDHHC8 is set forth in FIG. 11 (SEQ ID NO:19) and the wild-type amino acid sequence of ZDHHC8 is set forth in FIG. 10 (SEQ ID NO:18). As one non-limiting example, the presence of a mutation may be determined using techniques that evaluate the chromosomal DNA of a subject, such as Southern blotting and Restriction Fragment Length Polymorphisms (“RFLP” analysis). As another non-limiting embodiment, mRNA harvested from the subject may be tested to determine whether correctly spliced, wild-type transcript is present, for example, by Northern blot analysis or, to utilize a smaller mRNA sample, PCR analysis. In specific, non-limiting examples, PCR analysis may be performed to determine whether intron 4 is properly excised (for example, using primers from exons 1 and 8 (see Example 6, below) or another set of primers that span the exon 4-5 junction (e.g., primers derived from exons 2 and 6, 2 and 5, 3 and 6, 3 and 7, etc.), or to determine whether the DHHC motif is present, or to determine whether the wild-type carboxy terminus (exon 11) is present (using primers, for example, from the 3′ untranslated region and from an exon selected from exons 1-10 or, less preferably, 11). As another non-limiting embodiment, protein from the subject may be evaluated, for example by Western blot analysis or in an assay which determines whether protein collected from the subject can bind to and/or palmitoylate and/or translocate PSD93 and/or PSD95.

The foregoing may be determined using a sample from the subject which may be any suitable clinical sample, including but not limited to a blood sample (e.g., collection of peripheral blood lymphocytes), a tissue sample (e.g., epithelial cells), fetal cells obtained through amniocentesis or chorionic villus sampling, sperm cells, etc. To measure PSD93 or PSD95 palmitoylation the sample may be a brain biopsy.

Thus, the present invention may be used to identify an individual at risk of developing schizophrenia. Subjects identified as exhibiting decreased or absent expression of ZDHHHC8; decreased activity of ZDHHC8 (e.g. palmitoyltransferase activity); decreased activity of PSD93 or PSD95 (e.g. mediating the effects of the neurotransmitter glutamine via the formation of protein scaffolding), especially PSD95; or decreased palmitoylation and/or membrane translocation of PSD93 or PSD95, especially PSD95, when compared to a subject not diagnosed with schizophrenia or a related disorder, may be encouraged to enter a pschiatric evaluation and/or treatment program that may result in prompter treatment of symptoms that may arise.

Further, the present invention may be used to aid in establishing a diagnosis of schizophrenia or a related disorder, where decreased or absent ZDHHC8 expression; decreased activity of ZDHHC8; decreased activity of PSD93 or PSD95, especially PSD95; or decreased palmitoylation and/or membrane translocation of PSD93 or PSD95, especially PSD95, when compared to a subject not diagnosed with schizophrenia or a related disorder, is supportive of a diagnosis of schizophrenia or a related condition. Such a diagnostic test may further be used to direct a subject, having decreased or absent ZDHHC8 expression; decreased activity of ZDHHC8; decreased activity of PSD93 or PSD95, especially PSD95; or decreased palmitoylation and/or membrane translocation of PSD93 or PSD95, especially PSD95, when compared to a subject not diagnosed with schizophrenia or a related disorder, to a treatment regimens which compensates for that defect, for example, to a treatment regimen which increases levels of ZDHHC8; increases the activity of ZDHHC8; increases the activity of PSD93 or PSD95, especially PSD95; reintroduces a ZDHHC8, gene, or promotes palmitoylation/membrane translocation of PSD93 or PSD95, especially PSD95.

### 5.2 Transgenic Animals

The present invention provides for a transgenic non-human animal in which expression of at least one copy of endogenous ZDHHC8 is decreased or prevented. In preferred embodiments, expression of both copies of endogenous ZDHHC8 is decreased or prevented, and most preferably, detectable expression of the active protein is absent. In specific, non-limiting embodiments, the transgenic animal is a “knock-out” mouse which is homozygous or heterozygous for a knock-out mutation. Such a mouse may be prepared using standard laboratory techniques.

The present invention further envisions a “knock-out” transgenic animal, in which endogenous ZDHCC8 is expressed at very low or undetectable levels, into which a heterologous ZDHCC8-encoding transgene is introduced. The transgene may be from the same or a different species as its transgenic host. The ZDHCC8 may be wild-type or mutant gene. The transgene may be operably linked to a promoter element, which may be, for example and not by way of limitation, a constitutively active promoter, a neuron specific promoter, and/or an inducible promoter.

Such transgenic animals may be used as experimental models of schizophrenia and related disorders and may be used to identify agents useful in treating schizophrenia and/or related disorders (see below).

### 5.3 Assay Systems

The present invention provides for methods and assay systems for identifying agents that may be used to treat schizophrenia or a related disorder in a subject carrying a mutation that decreases or prevents ZDHHC8 expression. As one non-limiting example, a test agent may be administered to a transgenic animal in which expression of one or both copies of ZDHHC8 is decreased or prevented, and the animal may be tested for behavior, such as spontaneous exploratory behavior, (e.g., total distance traveled, or (for rodents) number of vertical moves, or entry into centerfield), where an increase in such behavior bears a positive correlation with the ability of the test agent to treat schizophrenia or a related disorder. In an analogous test in rodents, a test agent which may have therapeutic benefit would increase prepulse inhibition scores (see Example 6, below, for an assay for prepulse inhibition of the acoustic startle response). In preferred, non-limiting embodiments, the transgenic animal is a female, as females tend to exhibit mutant phenotypes more than males.

In another non-limiting example, a cell in which endogenous ZDHHC8 expression is decreased or prevented, or activity is decreased, may be exposed to a test agent, and the level of PSD93 or PSD95 activity, palmitoylation, and/or membrane translocation may be measured, where an increase in the activity, palmitoylation, and/or membrane translocation of either PSD93 or PSD95, especially PS95, bears a positive correlations with the ability of the test agent to treat schizophrenia or a related disorder. The test cell may be a human or a non-human cell, and may be a neuronal or non-neuronal cell.

In a specific non-limiting embodiment of the invention, the cell is a human cell which is heterozygous or homozygous for a mutation that disrupts ZDHHC8 expression, into which a fusion protein comprising PSD95 linked to a fluorescent protein, such as GFP, YFP, BFP, RFP, or CFP. A test agent is administered to said cell, and the cell is evaluated for the effect of test agent on membrane translocation of PSD95, using immunocytochemistry and/or cellular fractionation. The ability of a test agent to increase membrane translocation of PSD95 relative to negative control (absence of test agent) correlates positively with the ability of the test agent to treat schizophrenia or a related disorder. A test agent having such a positive result may then optionally be subjected to further testing, for example, in a transgenic animal having a homozygous or heterozygous mutation in ZDHHC8 expression, where an increase in exploratory behavior or an increase in prepulse inhibition score further positively correlates with therapeutic efficacy.

In an alternative non-limiting embodiment of the invention, an assay may utilize a cell which is a human cell which is heterozygous or homozygous for a mutation that disrupts ZDHHC8 expression. A test agent may be administered to the cell, and the amount of PSD95 palmitoylation may be measured and compared to the amount in a negative control cell. The ability of the test agent to increase palmitoylation correlates positively with the ability of the test agent to treat schizophrenia or a related disorder. A test agent having such a positive result may then optionally be subjected to further testing, for example, in a transgenic animal having a homozygous or heterozygous mutation in ZDHHC8 expression, where an increase in exploratory behavior or an increase in prepulse inhibition score further positively correlates with therapeutic efficacy.

### 5.4 Methods of Treatment

The present invention provides for a method of treating a subject suffering from schizophrenia or a related disorder comprising administering, to the subject, an effective amount of an isolated nucleic acid encoding human ZDHHC8, operably linked to a suitable promoter, optionally comprised in a suitable vector molecule. Suitable promoters include, but are not limited to, constitutively active promoters, neuron specific promoters (e.g., neuron specific enolase promoter), and inducible promoters (e.g., tetracycline inducible promoters). Suitable vectors include, but are not limited to, adenovirus, adeno-associated virus, retrovirus, vaccinia virus, etc. As a non-limiting example, ZDHHC8-encoding nucleic acids which may be used according to the invention include a human ZDHHC8 nucleic acid encoding a protein having a sequence as set forth in GenBank Acc. No. NM_{—}013373 and FIG. 10, or a nucleic acid having a sequence as set forth in GenBank Acc. No. NM_{—}013373 and FIG. 11, or a nucleic acid that is at least about 85, at least about 90, or at least about 95 percent homologous thereto, as determined by homology determining software such as BLAST or FASTA.

The present invention further provides for methods of treating a subject suffering from schizophrenia or a related disorder comprising administering, to the subject, an agent which increases the level of palmitoylated PSD93 and/or PSD95, such as Palmitoyl Coenzyme A, lecithin, or an isolated nucleic acid comprising a nucleic acid encoding human Zdhhc8, operably linked to a suitable promoter.

### 6. EXAMPLE

### Evidence that the Gene Encoding ZDHHC8 Contributes to the Risk of Schizophrenia

### 6.1 Materials and Methods

This working example was essentially published as Mukai J. et al. Evidence that the gene encoding ZDHHC8 contributes to the risk of schizophrenia. Nature Genetics 36, 725-731, published less than a year prior to the filing of this provisional application, and incorporated by reference in its entirety herein.

Construction and analysis of ZDHHC8 minigenes. Primer pair F6-ZDHHC8, R6-ZDHHC8 (TABLE 3, at the end of this section) was used for amplification of the genomic sequence surrounding the alternatively spliced exons 4 and 5 of ZDHHC8. Human genomic DNA homozygous for AA or GG at SNP rs175174 was used as PCR template. The forward primer was tailed with EcoRV, while the reverse primer was tailed with NotI to facilitate cloning. PCR product was purified using Qiaquick PCR Purification Kit (Qiagen). The purified PCR product was then double digested with EcoRV and NotI (New England Biolabs), purified using Qiaquick Gel Purification Kit and ligated into vector pcDNA3.1 (Invitrogen), which was pre-digested with EcoRV and NotI. The ligation product was then transformed into DH5α. Plasmids isolated from the transformants were sequenced to confirm the ZDHHC8 genomic sequence and the genotypes for SNP rs175174. Plasmids of ZDHHC8 minigene constructs were purified using Endofree Plasmid Maxi Kit (Qiagen) and transfected to 293 cells using Lipofectamine 2000 transfection reagent (Life Technologies). 293 cells were maintained in Dulbecco's modified Eagle's medium (DMEM) supplemented with 10% fetal calf serum, 100 U/ml penicillin, 100 μg/ml streptomycin and 2 mM L-glutamine at 37° C. under humidified atmosphere containing 5% CO₂. One day before the transfection, 5×10⁵ cells were plated on a 35 mm×10 mm cell culture dish (Corning Incorporated) in the growth medium without antibiotics. On the day of the transfection, 4 μg DNA diluted in 250 μl DMEM was combined with 10 μl LipofectAmine-2000 (Invitrogen) diluted in 250 μl DMEM. The mixture was then incubated at room temperature for 20 min and added to the cell culture dish. The cells were further incubated at 37° C., 5% CO₂ for 36-48 h. Total RNA from the transfected cells was purified using RNeasy Mini Purification kit according to the manufacturer's instructions (Qiagen).

Quantification of the ZDHHC8 minigene expression. For semi-quantitative polyacrylamide gel electrophoresis, PCR was performed using Herculase™Enhanced DNA Polymerase following the manufacturer's instructions (Stratagene), except that [α-³²P]dCTP was used instead of dCTP to label the PCR product. A touchdown protocol was used for the amplifications, consisting of denaturation at 94° C.×3 min, 15 cycles of 94° C.×30 sec. 68° C. (minus 1° C./cycle)×45 sec, 72° C.×45 sec, 5 cycles of 94° C.×30 sec, 53° C.×45 sec, 72° C.×45 sec, and a final extension cycle of 72° C.×7 min. The PCR products were separated on 4-12% polyacrylamide gels (Invitrogen). The gels were then exposed to KODAK X-OMAT AR film and the band densities were quantified by using MCID-4.0 (Imaging Research Inc.). For real-time quantitative (Taqman) PCR assay, the total RNA was transcripted by using Taqman Reverse Transcription Reagents (Applied Biosystems). A random hexamer was used as the primer for reverse transcription. The transcripts from the minigene were amplified by using Taqman Universal PCR Master Mix following the manufacturer's instructions (Applied Biosystems). The quantitative PCR was performed in an ABI Prism 7700 Sequence Detection System (Applied Biosystems).

Cell culture and transfection. Primary cultures of hippocampal neurons were obtained from mice embryos at embryonic day 15-16. The entire hippocampus was isolated and dissociated with trypsin treatment and trituration, and cells were plated on poly-L-lysine-coated 12 mm glass coverslips at a cell density of 4×10⁴. Neurons were maintained in Neurobasal media (Gibco) supplemented with B-27 (Gibco), 0.5 mM L-glutamine, penicillin and streptomycin. For transfection of ZDHHC8 and YFP-PSD-95, hippocampal cultures were transfected with 0.5 μg of DNA using LipofectAmine-2000 in Neurobasal media supplemented with B-27 and 0.5 mM L-glutamine. HeLa cells were obtained from the American Type Culture Collection and maintained in DMEM supplemented with 10% fetal bovine serum. For transfection, HeLa cells (2×10⁴ per 12 mm cover slip) were transiently transfected with 0.5 μg of DNA using LipofectAmine-2000 in DMEM supplemented with 10% fetal bovine serum and cultured for 18 h. Expression vector for human ZDHHC8 was constructed by PCR amplification of ZDHHC8 coding sequence using the oligonucleotide pair: 5′-CGGAATTCGCCGCCACCATGCCCCGCAGCCCC (SEQ. ID. NO. 20) and 5′-CGGGATCCTATCACACCGAGATCTCGTAGGTGGT (SEQ. ID. NO. 21), digestion with EcoRI/BamHI, and ligation of the resulting fragment into EcoRI/BamHI-digested pcDNA3.1/Myc-His(−)C.

Immunocytochemistry. The 280GP antibody was obtained by immunization of guinea pigs with a synthetic peptide derived from the C-terminal sequence of human ZDHHC8 (KKVSGVGGTTYEISV) (SEQ. ID. NO.: 17). Pilot experiments indicated that 280GP recognizes only exogenously transfected protein with high specificity. For double-staining of ZDHHC8 and NMDA receptor, neurons were fixed with methanol for 10 min at −20° C., blocked with 1% BSA and 1% normal goat serum, and incubated with 280GP and mouse monoclonal antibody to NMDAR2B (BD Transduction Laboratories) at 1:200 dilutions for 1 h at room temperature. After several rinses with PBS, cells were incubated with the anti-guinea pig IgG-Cy3 (Jackson ImmunoResearch Laboratory; diluted 1:100 in blocking solution) and anti-mouse Cy5 (Jackson ImmunoResearch Laboratory; diluted 1:100 in blocking solution) at room temperature for 30 min. For HeLa cells, cells were fixed with 3.7% paraformaldehyde for 30 min at room temperature, and permeabilized with 0.1% sodium citrate containing 0.1% Triton X-100 for 20 min on ice. After blocking with 1% BSA and 1% normal goat serum for 1 h at room temperature, cells were incubated with anti-EEA 1 mouse monoclonal antibody (BD Transduction Laboratories) at 1:50 dilutions, anti-mannose 6-phosphate receptor mouse monoclonal antibody (Affinity Bioreagents) at 1:100 dilutions, anti-GM130 mouse monoclonal antibody (BD Transduction Laboratories) at 1:100 dilutions, anti-golgin-97 mouse monoclonal antibody (Molecular Probes) at 1:100 dilutions, and anti-GRP-78 rabbit polyclonal antibody (Stressgen) at 1:100 dilutions as primary antibodies. After several rinses with PBS, cells were incubated with the anti-guinea pig IgG-Cy3, and anti-mouse IgG-Alexa Fluor 488 (Molecular Probes; diluted 1:100 in blocking solution), or anti-rabbit IgG-Alexa Fluor 488 (Molecular Probes; diluted 1:100 in blocking solution) at room temperature for 30 min. To-Pro-3 iodide (Molecular Probes) was used to visualize the nucleus. Coverslips were mounted on Vectashield (Vector Laboratory). Fluorescent images of cells were captured and analyzed on LSM510 Meta Confocal laser-scanning microscope (Zeiss).

Gross brain morphology. Four Zdhhc8 null and four wild-type littermate mice, 9-15 weeks of age, were used. Brains were post-fixed in 4% paraformaldehyde and transferred to 30% sucrose at 4° C. until they sank. Using a sliding microtome, 40 mm coronal sections were obtained and one series of every four sections through the entire brain was Nissl-stained with cresyl violet (Sigma). Regions of interest were digitally photographed (Spot RT Camera, Diagnostic Instruments, Inc.) at 40× and 100× magnification (Nikon E800 microscope). The photographs were analyzed using NIH Image program (1.63).

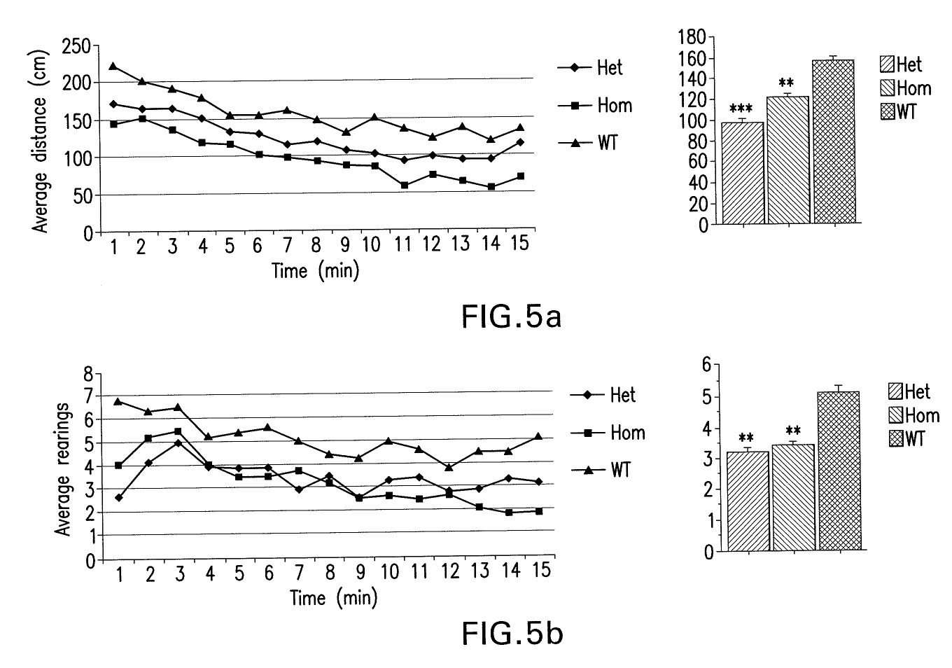

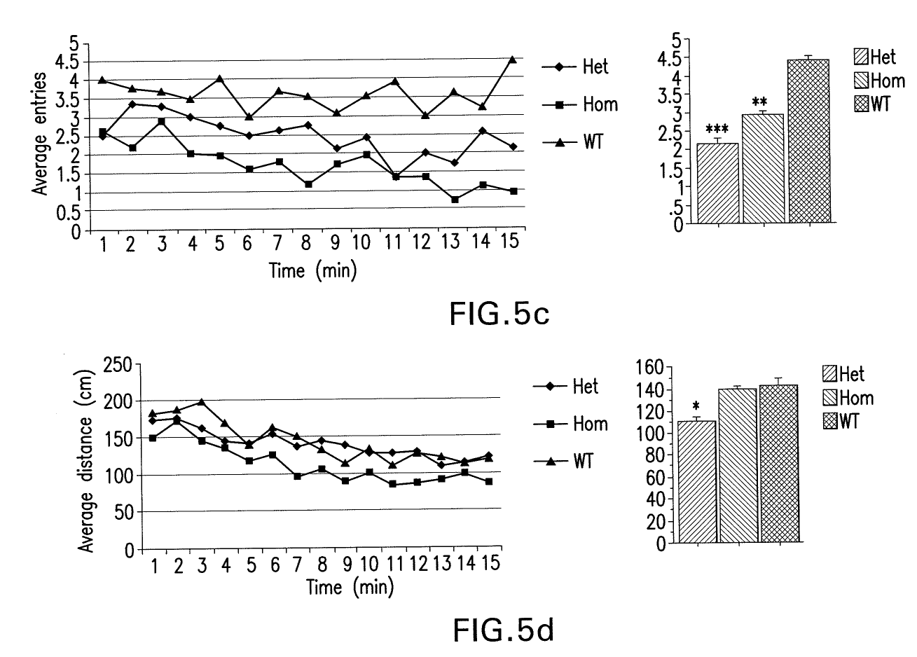

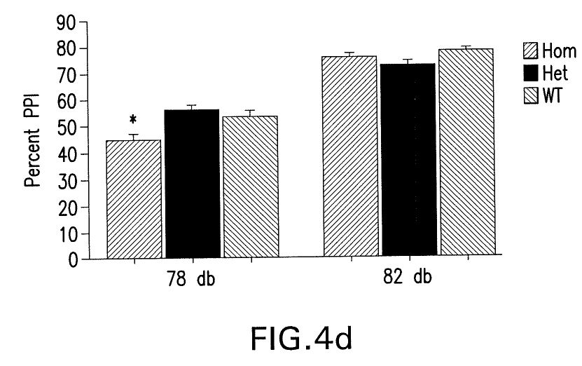

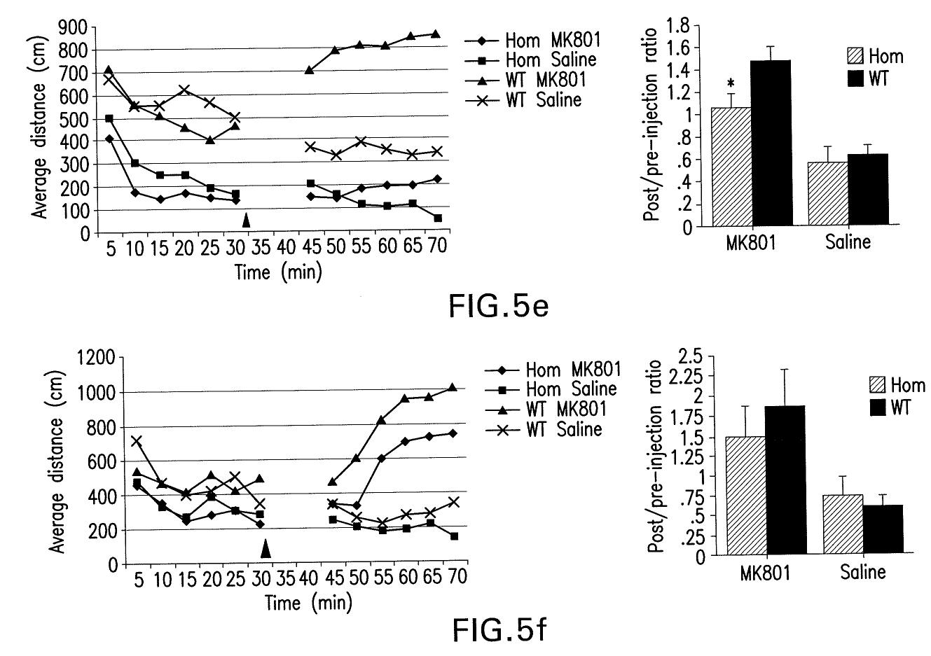

Generation of the Zdhhc8 knockout mice. Homologous recombination in ES cells was performed as described previously (39) using standard homologous recombination methods (see FIG. 5). The mutation was on a hybrid C57BL/6×129Sv background. Approximately 5% of the tested embryonic stem cell clones were positive for homologous recombination, and two clones were selected for injection into C57B6 blastocysts. Chimeric males were mated with C57BL/6 females, and DNA was genotyped from tail biopsy samples from F1 agouti-coat pups by Southern blotting and PCR at the Zdhhc8 locus. F1 heterozygous mice were mated to obtain F2 mice of all three genotypes. Zdhhc8 homozygous mutant mice were viable, fertile and had normal nesting behavior.

Assay for prepulse inhibition of the acoustic startle response. Adult (4-mo old) Zdhhc8 null and wild-type littermate mice were housed individually for 2 weeks prior to testing. Testing was conducted in a SR-Lab system (San Diego Instruments). Response amplitude was calculated as the maximum response level occurring during the 100 ms recording. Because animals can in principle habituate to the prepulse, as well as to the startle stimulus, the number of trials was kept to the essential minimum. Immediately after placement in the chamber, the animal was given a 5 min acclimation period during which background noise (67 dB) was continually present, and then received 8 sets of the following 4 trial types distributed pseudorandomly and separated by an average of 15 sec intertrial intervals: Trial 1: 40 ms, 120 dB noise burst alone; Trial 2-3: 120 dB startle stimuli preceded 100 ms by a 20 ms, 78 dB or 82 dB noise burst (prepulse); Trial 4: no-stimulus/background noise alone (67 dB). Data was analyzed using ANOVA repeated measures.

Open field assay. Animals were not pre-exposed to the chamber before testing. Activity was monitored in a clear acrylic chamber directly illuminated and equipped with infrared sensors for the automatic recording of horizontal and vertical activity (Colbourn Instruments). Each animal was placed initially in the periphery of the chamber, and recordings of the total distance traveled, the number of rearings (vertical moves) and the number of entries in the centerfield over the next 15 min were used as a measure of a locomotor response to novelty. 47 female mice (17 homozygous Zdhhc8 mutant, 17 heterozygous Zdhhc8 mutant and 13 wild-type littermate controls) and 45 male mice (13 homozygous Zdhhc8 mutant, 19 heterozygous Zdhhc8 mutant and 13 wild-type littermate controls) were tested.

Drug administration. MK801 was purchased from Sigma. Each experimental group consisted of 8-10 mice, which received the same number of injections, administered intraperitoneally, of either drug (0.4 mg per kg body weight) or vehicle. After 30 minutes of prehabituation in the open field, the mice were removed, injected and then left to rest for 10 minutes in their home cage before returning them to the open field arena, where they were monitored for an additional 30 minutes.

Fetal brain samples. Tissues were obtained from the tissue collection and distribution program at the University of Washington, Laboratory for the study of embryology. The tissues were from 90-120 day old specimens and were snap-frozen in liquid nitrogen.

Patient samples. Both our US and Afrikaner adult schizophrenia family samples have been described in detail elsewhere (19, 13). All methods were approved by Institutional Review Boards at participating sites and all participants signed appropriate informed consent. Allele transmission calculations were performed using the TDT method as described elsewhere (19, 13).

### 6.2 Results and Discussion

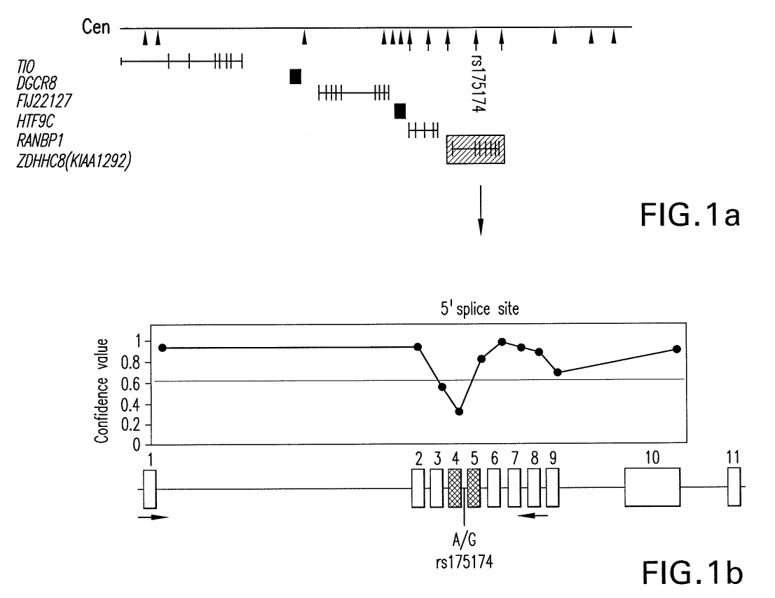

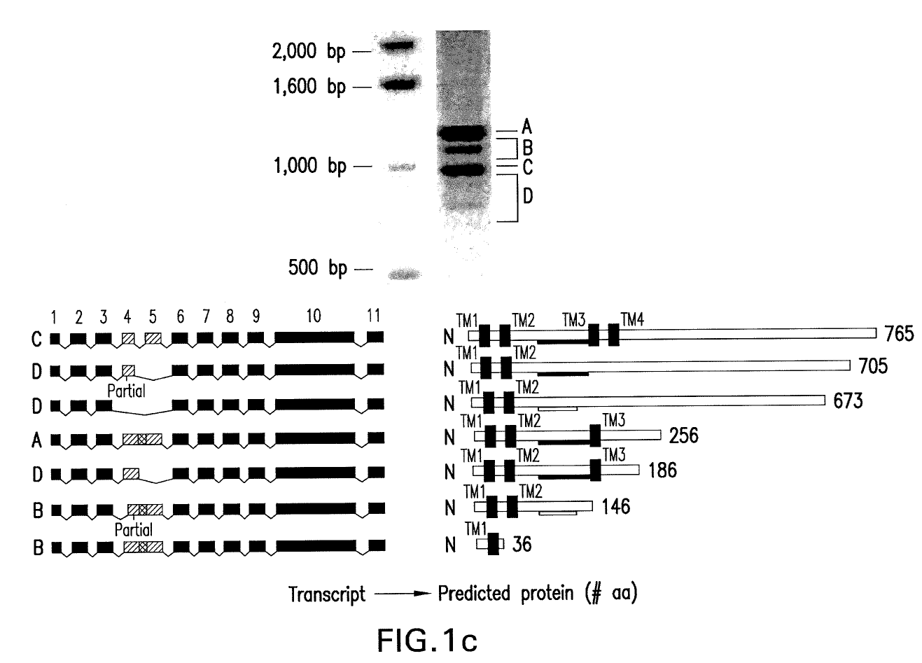

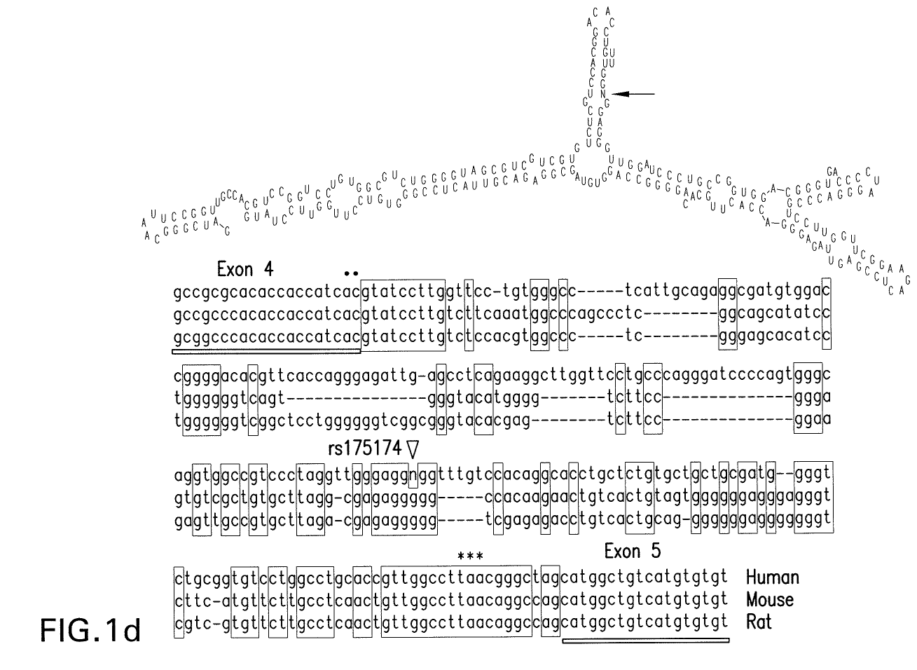

Association with schizophrenia in the distal part of the 22q11 locus implicated 5 neighboring SNPs distributed within a haplotypic block of 80 kb and having alleles with nominally significant association results (13; FIG. 1A). However, the equivalent statistical properties of SNPs within this block hindered the identification of the causal SNP on the basis of genetic evidence alone. Six known genes found in the RefSeq database are located in or immediately around the 80 kb haplotypic block (13; FIG. 1A). Extensive sequencing analysis of the coding region of these 6 genes failed to identify any linked causal missense allele (13). SNP rs175174, located in intron 4 of the ZDHHC8 gene (previously annotated as KIAA1292, FIG. 1A-B), shows the strongest and most consistent evidence of association with the disease from all 72 SNPs we tested from the 22q11 locus. SNP rs175174 showed significant association with schizophrenia in our original discovery sample of adult schizophrenics from the US (Pₙₒₘᵢₙₐₗ=0.0008, P_{empirical}=0.043 after permutation based correction for multiple tests), as well as in a family (P=0.007) and a case/control (P=0.05) sample from South Africa (13).

Preliminary analysis of EST databases identified alternatively spliced variants of the ZDHHC8 gene involving exons 4 and 5 (13). Therefore, a potential effect of rs175174 genotype on splicing warranted further investigation. Total or polyA+ RNA was prepared from fresh (non-transformed) human lymphocytes and subjected to reverse transcription and PCR amplification using primers located in exons 1 and 8. The PCR products were separated on a 1.2% agarose gel, purified and sequenced. This analysis revealed a highly reproducible, complex pattern of alternative splicing around intron 4. Interestingly, the two major products of comparable abundance were identified in both the total (FIG. 1C) and polyA+ RNA fraction, derived either from the fully spliced form (product A in FIG. 1C) or from an unspliced form retaining intron 4 (product C in FIG. 1C). Sequence analysis of the introns and their flanking regions revealed a suboptimal 5′ splice site (FIG. 1B) that can reduce the efficiency of splicing, leading to the retention of intron 4 (20, 21). Examples of intron retention have been found in transcripts from several species (22). It has been shown that suboptimal 5′ splice site can lead to accumulation of unspliced mRNA, which is able to enter the cytoplasm (21). The retained intron introduces a stop codon in the open reading frame that could lead to premature termination of translation (FIG. 1D). Therefore, the degree of intron 4 retention can regulate gene expression without changing transcriptional activity. Splicing of introns can be influenced by cis- or trans-acting mechanisms and cis-acting elements within an intron can reduce the efficiency of intron removal (20). SNP rs175174 is located at the middle of intron 4 embedded within a highly conserved heptanucleotide motif (FIG. 1D).

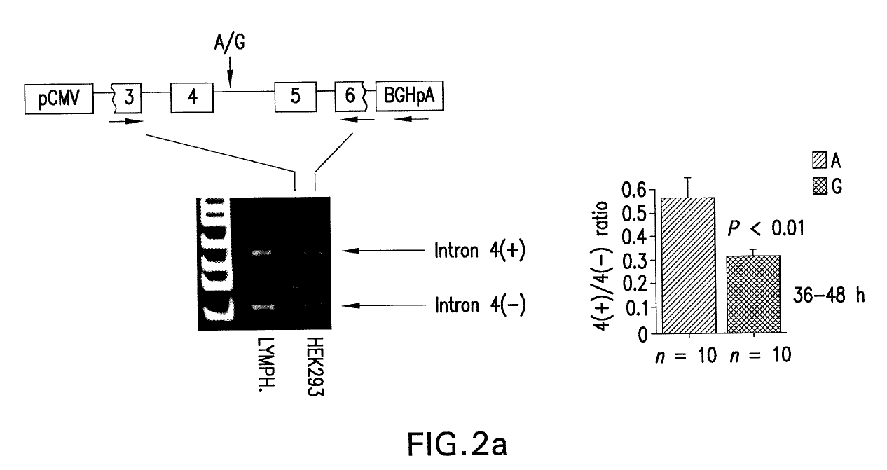

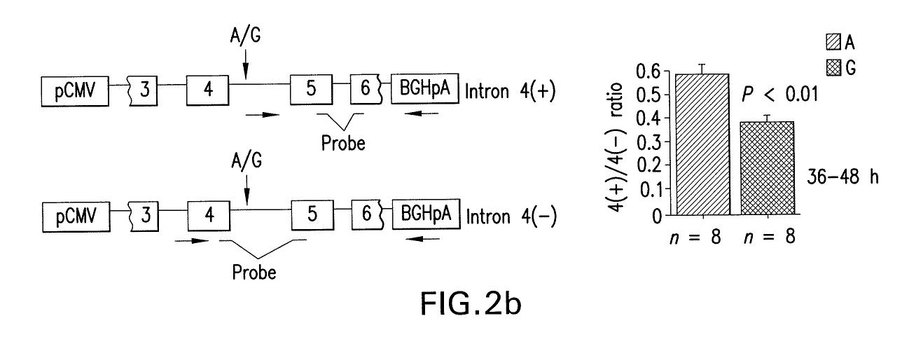

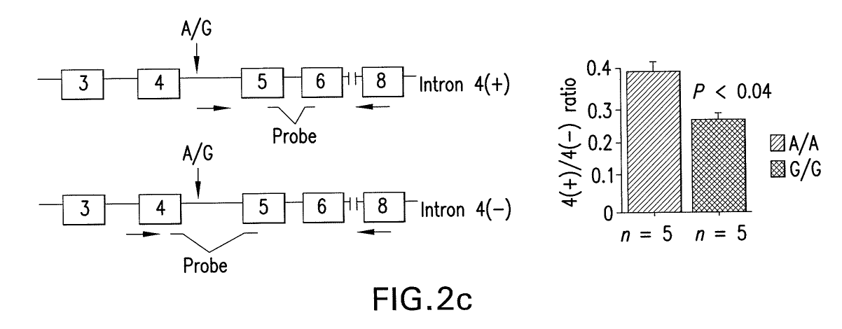

It was determined whether the genotype at SNP rs175174 affects the rate of intron 4 retention and therefore the ratio of the intron 4-containing splice form over the fully spliced form. Specifically, we examined whether presence of the risk allele A results in the production of relatively higher levels of the unspliced form. Minigenes from the “risk” and “non-risk” alleles containing exon 4, intron 4, exon 5, as well as flanking genomic sequences were constructed and transfected into 293 cells. Both minigenes were sequenced in their entirety and no variation beyond the rs175174 A/G was present. Initial semi-quantitative PCR amplification analysis revealed that the pattern of splicing observed in lymphocytes was recapitulated in total RNA prepared from 293 cells transiently transfected with the minigene (FIG. 2A) and that the relative amount of the intron 4-containing splice form is significantly increased in the RNA produced by the “risk” minigenes (FIG. 2A). Real-time quantitative RT-PCR analysis using a different set of primers confirmed this finding and allowed an accurate estimation of the observed increase at ˜33% (FIG. 2B). Based on our in vitro findings, we employed real-time quantitative RT-PCR on total fetal brain RNA and confirmed that the ratio of unspliced/spliced transcript was ˜25% higher in AA (risk allele) homozygotes as compared to GG (non-risk allele) homozygotes (P=0.04) (FIG. 2C). Demonstration that rs175174, which showed the strongest and most consistent evidence of association with schizophrenia of all 72 SNPs we tested from the 22q11 locus, can regulate the level of fully functional ZDHHC8 transcript strengthens the case of ZDHHC8 as an excellent candidate gene from this region and of SNP rs175174 as at least one of the causative variants.

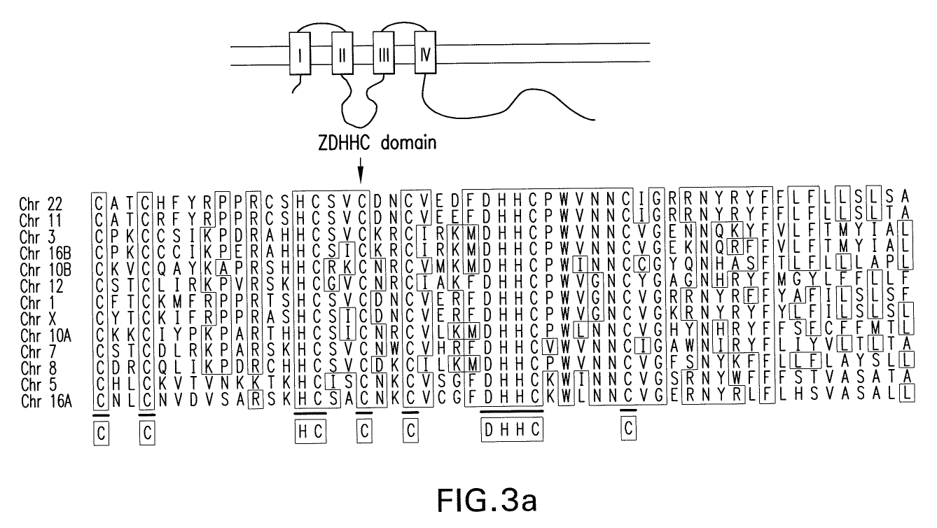

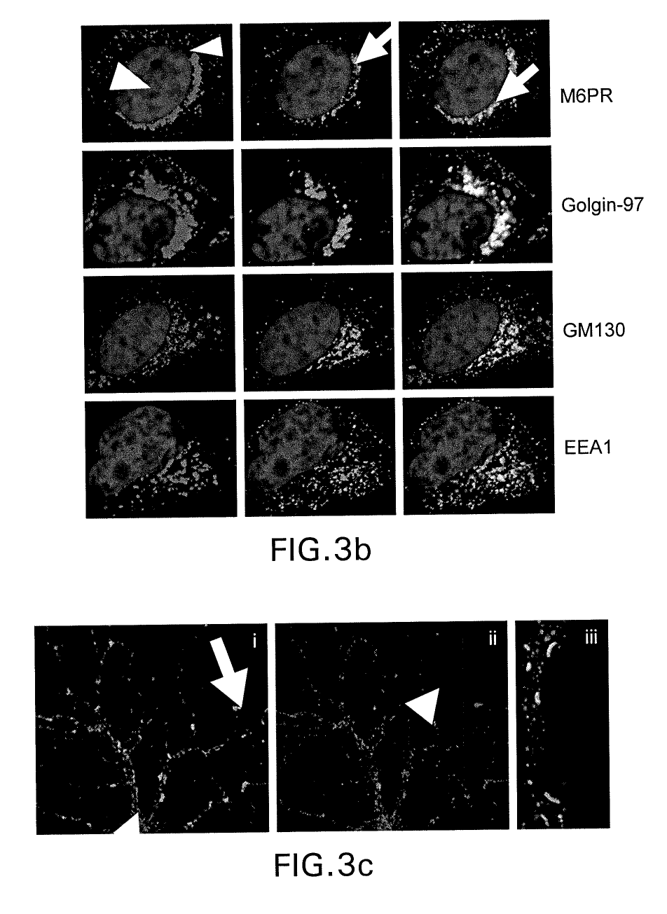

ZDHHC8 is predicted to have four transmembrane (TM) domains and a cysteine-rich domain that includes a DHHC motif and a Cys4 zinc-finger-like (Z) metal binding site (FIG. 3A). This region is largely part of the loop between adjacent TM2 and TM3. Homology searches found at least ten ZDHHC members in the human genome (FIG. 3A). Similar domains in open reading frames deduced from several species have been previously described (23-26). Two yeast ZDHHC proteins, ERF2 and AKR1 are transmembrane palmitoyltransferases (27, 28). ZDHHC8 expressed in HeLa cells and immunostained with an antibody raised against a C-terminal peptide, occurs throughout the cytoplasm in vesicle-like clusters but is also conspicuously concentrated at a perinuclear compartment. ZDHHC8 immunostaining shows partial overlap with the well-characterized cis-Golgi marker GM130, trans-Golgi marker golgin-97, late endosomal marker mannose 6-phosphate receptor and early endosomal marker EEA1 (FIG. 3B). No overlap was observed between ZDHHC8, the ER markers Grp78 and calnexin and the nuclear marker To-Pro-3 iodide (FIG. 3B). In primary developing hippocampal neurons transfected with ZDHHC8, immunoreactivity was found localized to a perinuclear domain, as well as to vesicle-like clusters along the dendritic shafts. Occasionally, ZDHHC8 immunoreactivity co-localizes along the processes with Post-Synaptic Density-95 (PSD-95) clusters at the developing dendritic spines (FIG. 3C).

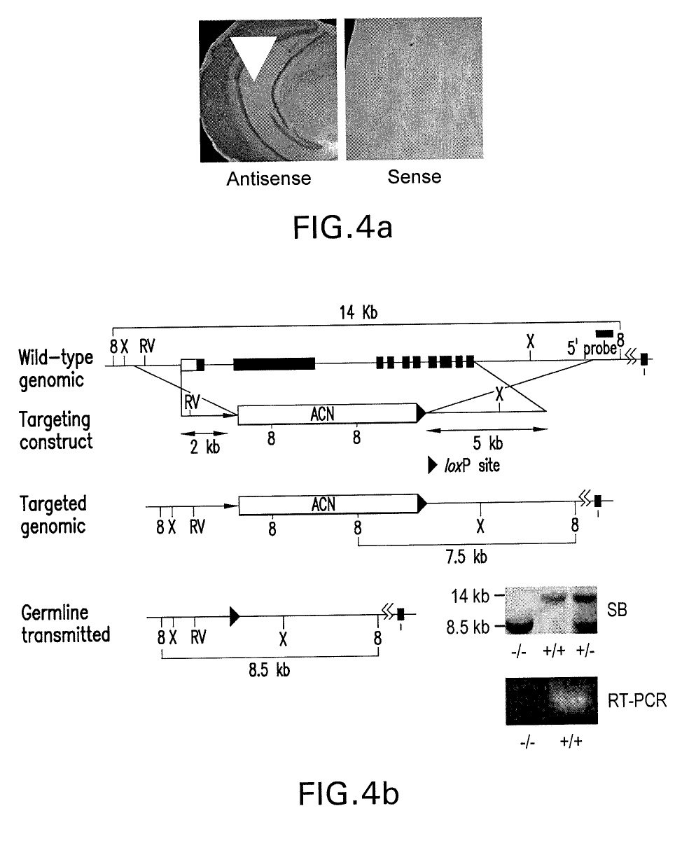

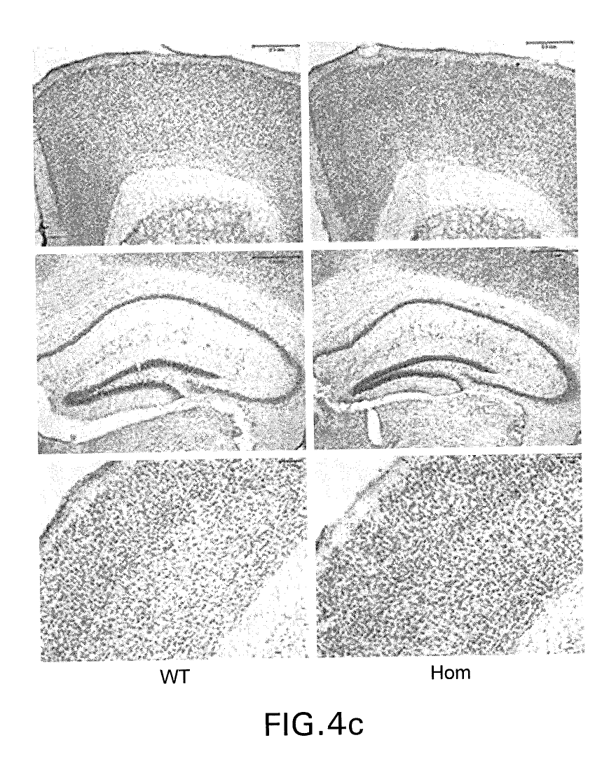

Recent work has shown that palmitate reversibly modifies numerous classes of neuronal proteins, including proteins important for neuronal development, neurotransmitter receptors and synaptic scaffolding proteins (29). ZDHHC8 is widely expressed in the adult human brain (13). More detailed analysis of expression in the adult mouse brain demonstrated higher expression levels in the cortex and hippocampus (FIG. 4A), areas presumed to play an important role in the pathophysiology of schizophrenia. In order to begin understanding how deficits in ZDHHC8 activity may affect neuronal development and/or synaptic transmission and eventually predispose to schizophrenia, homologous recombination was used to produce a knockout mouse model (FIG. 4B). Zdhhc8 null mice were viable, fertile and showed normal nesting behavior. Brain morphology appeared normal in Zdhhc8 null mice by gross evaluation and histological examination of sections (FIG. 4C) suggesting that impaired palmitoylation of ZDHHC8 substrates is not necessary for embryonic and postnatal brain development. The effect of ZDHHC8 deficiency in the state of sensorimotor gating was examined, one of the endophenotypes of schizophrenia widely studied in rodents (30, 17). An operational measure of sensorimotor gating is prepulse inhibition (PPI), the attenuation of a startle response by a weak pre-stimulus (prepulse) presented a short time (100 ms) prior to the startle stimulus. PPI was analyzed from adult (4-mo old) null mice and wild-type littermates of both sexes using combinations of one startle level (120 db) and two prepulse levels (78, 82 db). Consistent with a role of ZDHHC8 in schizophrenia susceptibility, female null mice (but not male) had significantly lower levels of prepulse inhibition compared to wild-type controls at 78 db (P=0.01) (FIG. 4D). No significant decreases in startle response amplitudes were identified. Further analysis of female null mice revealed additional deficits in locomotor activity in an open field test probing spontaneous exploratory behavior under mildly stressful conditions, that is, novel environment and light. Specifically, both homozygous and heterozygous Zdhhc8 mutant female mice traveled significantly less distance (P=0.0001 and 0.004, respectively, FIG. 5A) and had significantly fewer rearing responses (P=0.001 and 0.005, respectively; FIG. 5B) than their wild-type littermates in the open field assay. Moreover, both homozygous and heterozygous Zdhhc8 mutant female mice entered the aversive centerfield significantly less frequently that their wild-type littermates (P=0.0005 and 0.002, respectively; FIG. 5C) and moved significantly less in the center (about 2 times less). These results indicate that a greater fear of new environments probably accounts for the decreased locomotor activity. The effect of ZDHHC8 deficiency in exploratory behavior seems to be sexually dimorphic, as homozygous Zdhhc8 mutant male mice had only a modest deficit in locomotor activity in the open field (P=0.03) and heterozygotes did not differ from wild-type littermate controls in this respect (FIG. 5D).

Given the effects of ZDHHC8 deficit on behavior, it may be relevant that protein palmitoylation modulates several neurotransmitter systems (29), including activity-dependent plasticity at glutamatergic synapses (38). The effects of the psychomimetic dizocilpine (MK801), which is traditionally considered to be a NMDA-receptor blocker but probably also results in secondary activation of non-NMDA receptor glutamatergic neurotransmission by increasing glutamate efflux (41), in homozygous Zdhhc8 mutant mice and wild-type littermate controls. The ratio of activity used was during the half-hour after the injection to the activity during the half-hour before the injection as an index of the locomotor-activating effects of the drug. In terms of ratio index, homozygous Zdhhc8 mutant female (but not male) mice seemed to be somewhat less sensitive to MK801-induced stimulation of locomotor activity than wild-type littermate controls (P=0.05; FIG. 5E-F). This result suggests that ZDHHC8 affects behavior at least partly by interfering with glutamatergic transmission. In that respect, it is worth noting that the behavioral effects of MK801 seem to be sexually dimorphic in pharmacological animal models of schizophrenia symptoms also (42). Further analysis will be needed to dissect the neural pathways affected by ZDHHC8 deficiency.

Experiments were performed to determine whether a similar sexual dimorphism exists between functional variants of the ZDHHC8 gene and schizophrenia in humans. SNP rs175174 was examined in an extended sample of 389 parent-proband triads from the US and the Afrikaner population of South Africa that includes the families described in the original study. Indeed, in the extended sample of nuclear families the presence of a sexually dimorphic effect was confirmed that was also initially observed in the discovery sample (13; TABLE 2, at the end of this section). Specifically, analysis of allele transmission by gender revealed a, striking sex-related heterogeneity of transmission (heterogeneity χ²=8.2, P=0.004). The effect of SNP rs175174 was found to be highly significant in females (allele A predisposing χ²=12.2, P=0.0005, T/U: 82/43). Males did not show any effect (χ²=0.02, P=0.88, T/U: 106/108). Interestingly, two recent studies involving large-scale collections of schizophrenic patients also identified striking female-specific effects for variants of two strong candidate schizophrenia susceptibility genes (32, 33), including the gene for COMT also located within the 22q11 microdeletion.

An emerging “genetic” picture is that the 22q11 microdeletion-associated schizophrenia may have the characteristics of a contiguous gene syndrome where more than one gene may contribute to the dramatic increase in disease risk (13). Systematic approaches as well as candidate gene studies designed to identify schizophrenia susceptibility genes from the 22q 11 region have implicated so far the genes encoding PRODH (20, 34, 35), COMT (32, 36) and ZDHHC8 (13 and herein). Recent studies in animal models (17) have started addressing potential interactions between these genes. These studies have shown that PRODH deficiency (that likely results in dysregulation of glutamate transmission (17, 36)) leads to a secondary dopaminergic hyperresponsivity and subsequent compensatory changes that include increase in the levels of COMT-mediated dopamine breakdown. Therefore, deficiency of the 22q11 schizophrenia susceptibility genes modulates the disease risk either by impairing synaptic function or by failing to compensate for such impairment. Such synergistic interaction could in principle lead to the unprecedented increase in schizophrenia risk associated with microdeletions of this locus. It is of considerable interest that protein palmitoylation can modulate several neurotransmitter systems (29, 37) and, most notably, as shown in recent studies, can regulate synaptic strength and activity-dependent plasticity at glutamatergic synapses (38).

TABLE 2
Transmission of the risk allele rs175174-A

| Sample (# families) | Sex | T | U | P-value |
|---|---|---|---|---|
| Discovery (N = 106) | F | 30 | 10 | 0.0016 |
| | M | 35 | 22 | 0.08 |
| Total (N = 389) | F | 82 | 43 | 0.0005 |
| | M | 106 | 108 | 0.88 |

T = Transmitted; U = Untransmitted

TABLE 3
DNA sequences of primers and probes used in this study

| | | |
|---|---|---|
| Primer^{a} | Sequence^{b} | Location |

| | | |
|---|---|---|
| F1-ZDHHC8 1 | 5′-cag ccc cgg gac gcg cct caa ac-3′ | exon 1 |
| | (SEQ. ID. NO.: 22) | |
| R1-ZDHHC8 | 5′-tgg ccc caa gtc cag tgg ctt ctc-3′ | exon 8 |
| | (SEQ. ID. NO.: 23) | |
| F2-ZDHHC8 | 5′-tgg tgt gcc acg tgc cac ttc tac c-3′ | exon 3 |
| | (SEQ. ID. NO.: 24) | |
| R2-ZDHHC8 | 5′-cat tcc cac agc agc ctc ggg tga aag-3′ | exon 6 |
| | (SEQ. ID. NO.: 25) | |
| F3-ZDHHC8 | 5′-tgg tgt gcc acg tgc cac-3′ | exon 3 |
| | (SEQ. ID. NO.: 26) | |
| F4-ZDHHC8 | 5′-cct ggt cta cgt gct gaa-3′ | exon 4 |
| | (SEQ. ID. NO.: 27) | |
| F5-ZDHHC8 | 5′-ggt ttg tcc aca ggc acc-3′ | intron 4 |
| | (SEQ. ID. NO.: 28) | |
| R-BGHpA | 5′-tag aag gca cag tcg agg-3′ | BGHpA |
| | (SEQ. ID. NO.: 29) | |
| F6-ZDHHC8 | 5′-ccg gat atc tgg tgt gcc acg tgc cac ttc tac c-3′ | exon 3 |
| | (SEQ. ID. NO.: 30) | |
| R6-ZDHHC8 | 5′-aag gaa aaa agc ggc cgc att ccc aca gca gcc tcg ggt gaa ag-3′ | exon 6 |
| | (SEQ. ID. NO.: 31) | |
| Probe1-ZDHHC8 | 5′-6FAM-atc acc atg gct gtc atg tgt gtg g-TAMRA-3′ | exon 4/5 |
| | (SEQ. ID. NO.: 32) | |
| Probe2-ZDHHC8 | 5′-6FAM-cca acg agc agg tga ctg gga ag-TAMRA-3′ | exon 5/6 |
| | (SEQ. ID. NO.: 33) | |

^{a.}F, forward (top-strand) primer; R, reverse (bottom-strand) primer.
^{b.}Underlined sequences represent the restriction enzyme sites used for cloning.

### 7. EXAMPLE

### PSD95 and PSD93 are Substrates of ZDHHC8

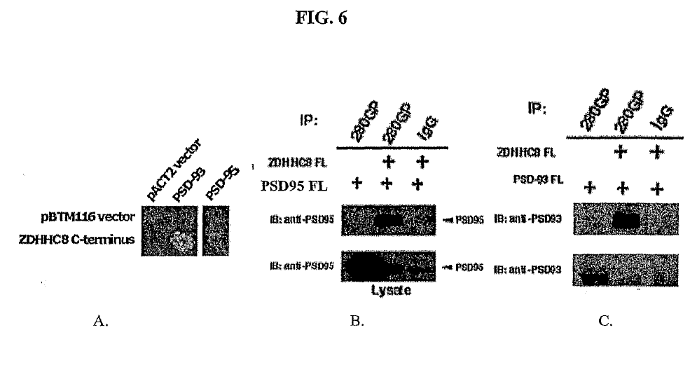

Using the yeast two-hybrid approach as well as co-immunoprecipitation assays (FIG. 6A-C), ZDHHC8 was shown to interact with two homologous proteins PSD95 and PSD93, both of which are key signaling molecules at a variety of synapses including glutamatergic synapses.

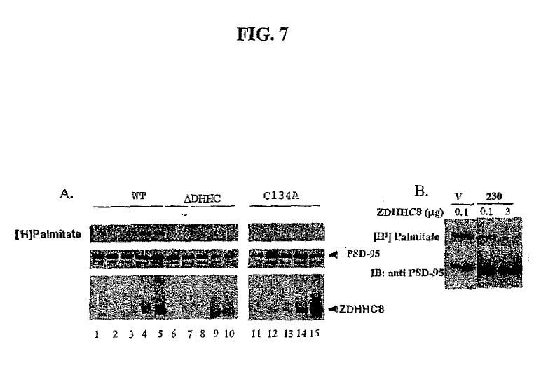

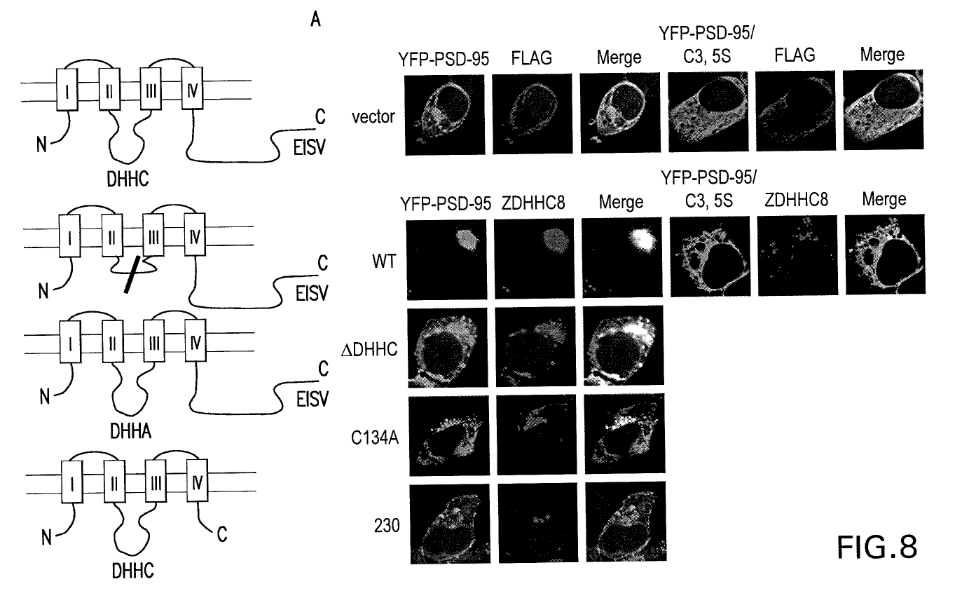

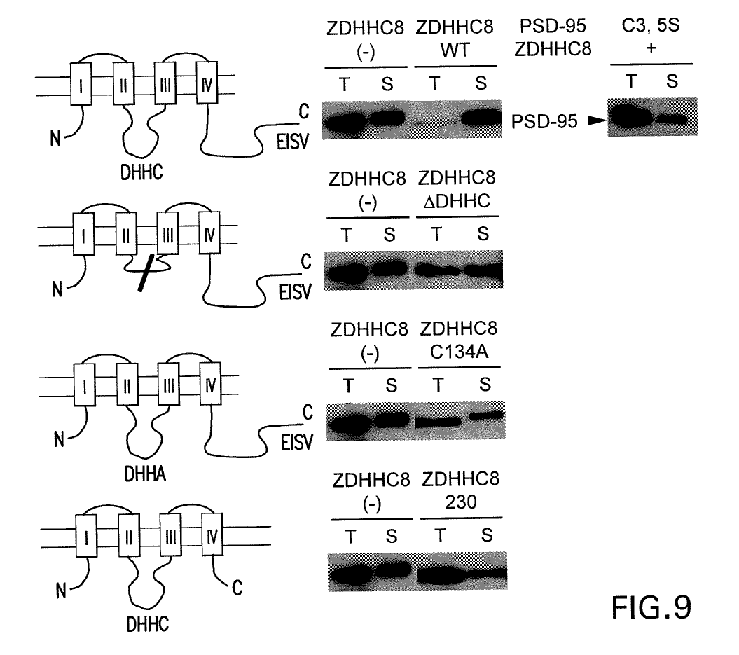

A series of ZDHHC8 mutants were prepared, either lacking the DHHC motif (“ΔDHHC”); carrying a mutation substituting cysteine for alanine at position 134, so that the DHHC motif become DHHA (“C134”); or lacking the C terminus (“230”). As shown in FIG. 7A-B, ZDHHC8 palmitoylates PSD95 in a manner that depends on an active catalytic domain and an intact C-terminus. In cells cotransfected with PSD95 fluorescently labeled with yellow fluorescent protein (“YFP”), it was shown that wildtype ZDHHC8 facilitated membrane translocation of PSD95; mutant forms of ZDHHC8 has substantially less translocation-facilitating activity, with the 230 mutant (lacking the C-terminal end) most severely impaired, as shown by both immunocytochemistry and cellular fractionation assays (FIG. 8 and FIG. 9, respectively). Of note, translocation also did not occur in the presence of wild-type ZDHHC8 where the palmitoylation site of PSD95 was mutated (PSD95-C3,5S) (FIG. 8 and FIG. 9).

The foregoing results are consistent with PSD95 being an authentic substrate of ZDHHC8. Activity-dependent palmitoylation of PSD95 has been known to affect plasticity at glutamatergic synapses. This is important because it is widely believed that impaired glutamate transmission underlies both the cognitive and negative symptoms of schizophrenia.

### 8. EXAMPLE

### PSD95 Palmitoylation by ZDHHC8 is Necessary for PSD95 Expression, GLUR2Clustering, And Dendritic Architecture

It is widely believed that impaired glutamate transmission underlies both the cognitive and negative symptoms of schizophrenia. Abnormal expression of Post-Synaptic Density (PSD)-proteins, which form a protein scaffold mediating the effects of the neurotransmitter glutamate, is thought to be involved in the pathophysiology of schizophrenia. Recently, it was shown that the level of one of these proteins (PSD95) was significantly decreased in the frontal cortex of individuals with schizophrenia (49).

We have shown that PSD95 is a substrate for ZDHHC8 palmitoyltransferase activity in vitro and in vivo. The palmitoylation of PSD-95 is critical for PSD95 clustering of AMPA receptors at excitatory glutamatergic synapses (38), and is dynamically regulated by synaptic activity, such that cycling of palmitate on PSD95 can contribute to aspects of synaptic plasticity (38). Accordingly, we analyzed the effects of Zdhhc8 deficiency on synaptic function in primary cultured hippocampal neurons. We found that the total protein expression of PSD95 and surface expression of GluR2 receptor were reduced in neurons from heterozygous as well as homozygous Zdhhc8-deficient mice, indicating that ZDHHC8 plays fundamental roles in controlling glutamate transmission, consistent with its role as a schizophrenia susceptibility gene.

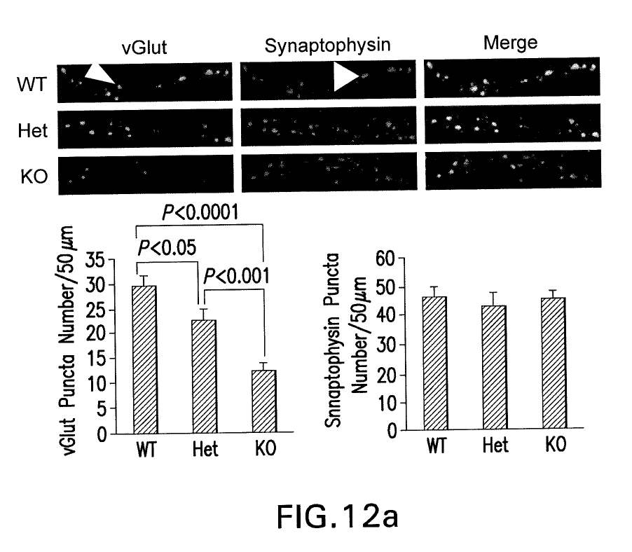

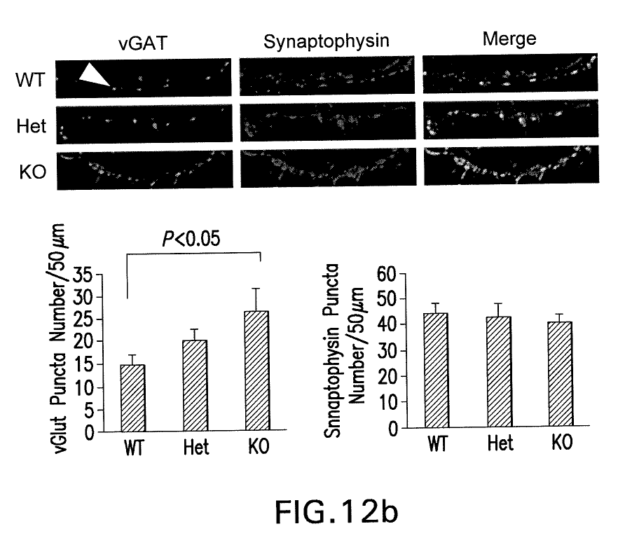

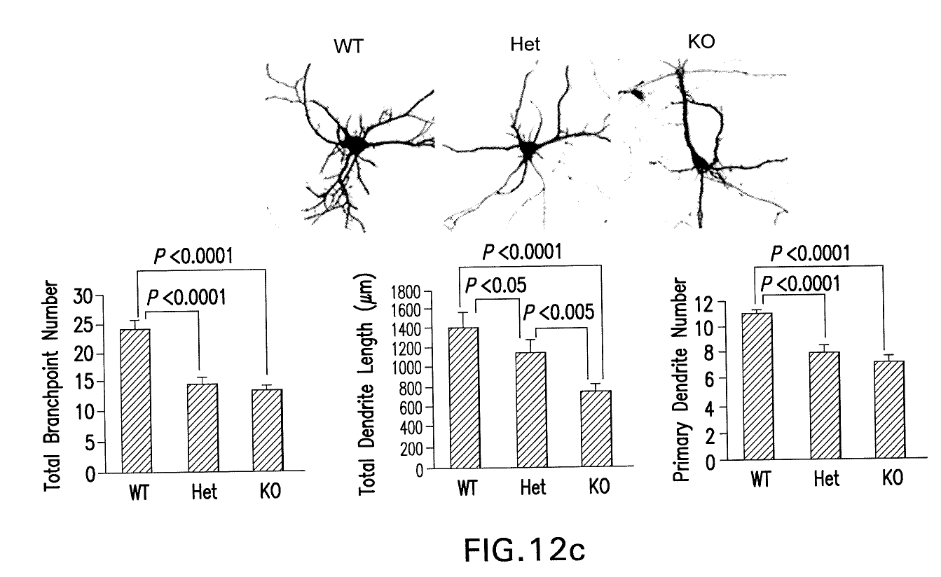

The balance between excitatory and inhibitory synapses is a tightly regulated process that requires differential recruitment of proteins that dictate the specificity of newly formed contacts. Decreased expression of PSD95 induced changes in the number of excitatory versus inhibitory contacts and results in overall decrease in the ratio of excitatory (glutamatergic) to inhibitory (GABAergic) synaptic currents (50). We found that the reduction of PSD95 induced by Zdhhc8 deficiency resulted in a decrease in the ratio of excitatory/inhibitory synaptic number in neurons from heterozygous as well as homozygous Zdhhc8-deficient mice (FIG. 12A-B). This reduction was reversed by introduction of a wild-type form of the Zdhhc8 gene, but not by a mutant form which lacks an enzymatic activity. Thus we have shown that the ZDHHC8 palmitoyltransferase activity is involved in the control of a balance between excitatory and inhibitory synapses via modulatating the levels of PSD95.

We also found that the deficiency in Zdhhc8 in hippocampal neurons affects dendritic tree morphogenesis (FIG. 12C). Specifically, Zdhhc8 deficiency results in decreases in the number of total branch points, total dendrite length, and number of primary dendrites, indicating that ZDHHC8 might be essential for emergence of normal dendritic architecture. Indeed, the dendritic architecture of neurons is markedly reduced in schizophrenia patients. (51, 52).

### 9. REFERENCES

1. Scambler, P. J., Hum. Mol. Genet. 9, 2421-2426 (2000).
2. Pulver, A. E., Nestadt, G., Goldberg, R., Shprintzen, R. J., Lamacz, M., Wolyniec, P S., Morrow, B., Karayiorgou, M., Antonarakis, S. E., Housman, D., et al., J. Nerv. Ment. Dis. 182, 476-478 (1994).
3. Murphy, K. C., Jones, L. A. & Owen, M. J., Arch. Gen. Psychiatry 56, 940-945 (1999).
4: American Psychiatric Association, Diagnostic and Statistical Manual of Mental Disorders (Am. Psychiatric Press, Washington, D.C.) (1994).
5. du Montcel, S. T., Mendizabal, H., Ayme, S., Levy, A. & Philip, N., J. Med. Genet. 33, 719 (1996).
6. Karayiorgou, M., Morris, M. A., Morrow, B., Shprintzen, R. J., Goldberg, R., Borrow, J., Gos, A., Nestadt, G., Wolyniec, P. S., Lasseter, V. K., et al., Proc. Natl. Acad. Sci. USA 92, 7612-7616 (1995).
7. Usiskin, S. I., Nicolson, R., Krasnewich, D. M., Yan, W., Lenane, M., Wudarsky, M., Hamburger, S. D. & Rapoport, J. L., J. Am. Acad. Child Adolesc. Psychiatry 38, 1536-1543 (1999).
8. Blouin, J. L., Dombroski, B. A., Nath, S. K., Lasseter, V. K., Wolyniec, P. S., Nestadt, G., Thornquist, M., Ullrich, G., McGrath, J., Kasch, L., et al., Nat. Genet. 20, 70-73 (1998).
9. Shaw, S. H., Kelly, M., Smith, A. B., Shields, G., Hopkins, P. J., Loftus, J., Laval, S. H., Vita, A., De Hert, M., Cardon, L. R., et al., Am. J. Med. Genet. 81, 364-376 (1998).
10. Bearden, C. E., Woodin, M. F., Wang, P. P., Moss, E., McDonald-McGinn, D., Zackai, E., Emannuel, B. & Cannon, T. D., J. Clin. Exp. Neuropsychol. 23, 447-464 (2001).
11., Chow, E. W., Mikulis, D. J., Zipursky, R. B., Scutt, L. E., Weksberg, R. & Bassett, A. S., Biol. Psychiatry 46, 1436-1442 (1999).
12. Chow, E. W., Zipursky, R. B., Mikulis, D. J. & Bassett, A. S., Biol. Psychiatry 51, 208-215 (2002).
13. Liu, H., Abecasis, G. R., Heath, S. C., Knowles, A., Demars, S., Chen, Y.-J., Roos J. L., Rapoport, J. L., Gogos, J. A., & Karayiorgou, M., Proc. Natl. Acad. Sci. U.S.A. 99(26), 16859-16864 (2002).
14. Edelmann, L., Pandita, R. K., Spiteri, E., Funke, B., Goldberg, R., Palanisamy, N., Chaganti, R. S., Magenis, E., Shprintzen, R. J. & Morrow, B. E., Hum. Mol. Genet. 8, 1157-1167 (1999).
15. Shaikh, T. H., Kurahashi, H., Saitta, S. C., O'Hare, A. M., Hu, P., Roe, B. A., Driscoll, D. A., McDonald-McGinn, D. M., Zackai, E. H., Budarf, M. L., et al., Hum. Mol. Genet. 9, 489-501 (2000).
16. Liu, H., Heath, S. C., Sobin, C., Roos, J. L., Galke, B. L., Blundell, M. L., Lenane, M., Robertson, B., Wijsman, E. M., Rapoport, J. L., Gogos, J. A. & Karayiorgou, M., Proc. Natl. Acad. Sci. USA 99, 3717-3722 (2002).
17. Gogos, J. A., Santha, M., Takacs, Z., Beck, K. D., Luine, V., Lucas, L. R., Nadler, J. V. & Karayiorgou, M., Nat. Genet. 21, 434-439 (1999).
18. Bassett, A. S. et al. The schizophrenia phenotype in 22q11 deletion syndrome. Am. J. Psychiatry 160, 1580-1586 (2003).
19. Liu, H. et al. Genetic variation at the 22q11 PRODH2/DGCR6 locus presents an unusual pattern and increases susceptibility to schizophrenia. Proc. Natl. Acad. Sci. USA 99, 3717-3722 (2002).
20. Dirksen, W. P., Sun, Q. & Rottman, F. M. Multiple splicing signals control alternative intron retention of bovine growth hormone pre-mRNA. J. Biol. Chem. 270, 5346-5352 (1995).
21. Huang, Y. & Carmichael, G. G. A suboptimal 5′ splice site is a cis-acting determinant of nuclear export of polyomavirus late mRNAs. Mol. Cell. Biol. 16, 6046-6054 (1996).
22. Kienzle, N. et al. Intron retention may regulate expression of Epstein-Barr virus nuclear antigen 3 family genes. J. Virol. 73, 1195-1204 (1999).
23. Linder, M. E. & Deschenes, R. J. New insights into the mechanisms of protein palmitoylation. Biochemistry 42, 4311-4320 (2003).
24. Uemura, T., Mori, H. & Mishina, M. Isolation and characterization of Golgi apparatus-specific GODZ with the DHHC zinc finger domain. Biochem. Biophys. Res. Commun. 296, 492-496 (2002).
25. Li, B., Cong, F., Tan, C. P., Wang, S. X. & Goff, S. P. Aph2, a protein with a zf-DHHC motif, interacts with c-Abl and has pro-apoptotic activity. J. Biol. Chem. 277, 28870-28876 (2002).
26. Singaraja, R. R. et al. HIP 14, a novel ankyrin domain-containing protein, links huntingtin to intracellular trafficking and endocytosis. Hum. Mol. Genet. 11, 2815-2828 (2002).
27. Roth, A. F., Feng, Y., Chen, L. & Davis, N. G. The yeast DHHC cysteine-rich domain protein Akr1p is a palmitoyl transferase. J. Cell. Biol. 159, 23-28 (2002).
28. Lobo, S., Greentree, W. K., Linder, M. E. & Deschenes, R. J. Identification of a Ras palmitoyl-transferase in Saccharomyces cerevisiae. J. Biol. Chem. 277, 41268-41273 (2002).
29. El-Husseini, A.el-D. & Bredt, D. S. Protein palmitoylation: a regulator of neuronal development and function. Nat. Rev. Neurosci. 3, 791-802 (2002).
30. Swerdlow, N. R. & Geyer, M. A. Using an animal model of deficient sensorimotor gating to study the pathophysiology and new treatments of schizophrenia. Schizophr. Bull. 24, 285-301 (1998).
31, Shifman, S., et al. A highly significant association between a COMT haplotype and schizophrenia. Am. J. Hum. Genet. 71, 1296-1302 (2002).
32. Hennah, W. et al. Haplotype transmission analysis provides evidence of association for DISC 1 to schizophrenia and suggests sex-dependent effects. Hum. Mol. Genet. 12, 3151-3159 (2003).
33. Jacquet, H. et al. PRODH mutations and hyperprolinemia in a subset of schizophrenic patients. Hum. Mol. Genet. 11, 2243-2249 (2002).
34. Collier, D. A. & Li, T. The genetics of schizophrenia: glutamate not dopamine? Eur. J. Pharmacol. 480, 177-184 (2003).
35. Egan, M. F. et al. Effect of COMT Val108/158 Met genotype on frontal lobe function and risk for schizophrenia. Proc. Natl. Acad. Sci. USA 98, 6917-6922 (2001).
36. Renick, S. E. et al. The mammalian brain high-affinity L-proline transporter is enriched preferentially in synaptic vesicles in a subpopulation of excitatory nerve terminals in rat forebrain. J. Neurosci. 19, 21-33 (1999).
37. Kanaani, J. et al. A combination of three distinct trafficking signals mediates axonal targeting and presynaptic clustering of GAD65. J. Cell Biol. 158, 1229-1238 (2002).
38. El-Husseini, A.el-D. et al. Synaptic strength regulated by palmitate cycling on PSD-95. Cell 108, 849-863 (2002).
39. Gogos, J. A., Osborne, J., Nemes, A., Mendelsohn, M. & Axel, R. Genetic ablation and restoration of the olfactory topographic map. Cell 103, 609-620 (2000).
40. Bunting, M., Bernstein, K. E., Greer, J. M., Capecchi, M. R. & Thomas, K. R. Targeting genes for self-excision in the germ line. Genes & Dev. 13, 1524-1528 (1999).
41. Moghaddam B. and Adams, B. Reversal of phencyclidine effects by a group II metabotropic glutamate receptor agonist in rats. Science 281:1349-1352 (1998).
42. D'Souza, D. N. et al. Sexual dimorphism in the response to N-methyl D-aspartate receptor antagonists and morphine on behavior and c-Fos induction in the rat brain. Neuroscience 93, 1539-1547 (1999).
43. Bateman, J. F. et al. Tissue-specific RNA surveillance? Nonsense-mediated mRNA decay causes collagen X haploinsufficiency in Scmid metaphyseal chondroplasia cartilage. Human Mol. Genet. 12, 217-225 (2003).
44. Drysdale C. M. et al., Complex promoter and coding region of beta-2 adrenergic receptor haplotypes alter receptor expression and predict in vivo responsiveness. Proc. Natl. Acad. Sci. U.S.A. 97, 10483-10488.
45. Peltekova, V. D. et al., Functional variants of OCTN cation transporter genes are associated with Crohn disease. Nature Genet. 36, 471-475 (2004).
46. Aleman, A. et al., Sex differences in the risk of schizophrenia: evidence from meta-analysis. Arch. Gen. Psychiatry 60, 565-571 (2003).
47. Li et al., Evidence for association between novel polymorphisms in the PRODH gene and schizophrenia in a Chinese population. Published online May 24, 2004 (doi:10.1002/ajmg.b.30049).
48. Egan, M. F et al., Effect of COMT Val108/158 Met genotype on frontal lobe function and risk for schizophrenia. Proc. Natl. Acad. Sci. U.S.A. 98, 6917-6922.
49 Kristiansen, L. V., et al., Changes in NMDA receptor subunits and interacting PSD proteins in dorsolateral prefrontal and anterior cingulate cortex indicate abnormal regional expression in schizophrenia. Mol. Psychiatry. May 16, 2006 (Epublication ahead of print).
50. Prange, O. et al., A balance between excitatory and inhibitory synapses is controlled by PSD-95 and neuroligin. Proc. Natl. Acad. Sci. U.S.A. 101, 13915-20 (2004).
51. Kalus, P. et al., The dendritic architecture of prefrontal pyramidal neurons in schizophrenic patients. Neuroreport. 11, 3621-5 (2000).
52. Rosoklija, G., et al., Structural abnormalities of subicular dendrites in subjects with schizophrenia and mood disorders: preliminary findings. Arch Gen Psychiatry. 57, 349-56 (2000).
53. Carlin R. C. et al., Isolation and Characterization of Postsynaptic Densities from Various Brain Regions: Enrichment of Different Types of Postsynaptic Densities. J. Cell. Biol. 86, 831-843 (1980).
54. Mukai J. et al., Evidence that the Gene Encoding ZDHHC8 Contributes to the Risk of Schizophrenia. Nature Genetics. 36, 725-731 (2004).

Various publications are cited herein, the contents of which are hereby incorporated by reference in their entireties.

What is claimed is:

1. A method of diagnosing schizophrenia or a related disorder in a subject comprising identifying the presence of a mutation that decreases ZDHHC8 expression or ZDHHC8 protein activity in the subject when compared to ZDHHC8 expression or ZDHHC8 activity in a subject not diagnosed with schizophrenia or a related disorder, wherein the presence of the mutation indicates a susceptibility to schizophrenia or a related disorder.

2. The method of claim 1, wherein the mutation is selected from the group consisting of a point mutation, a deletion, a substitution, an insertion, and an inversion.

3. The method of claim 1, wherein the mutation results in improper excision of ZDHHC8 intron 4.

4. The method of claim 1, wherein the subject is a mammal.

5. The method of claim 4, wherein the mammal is a human.

6. The method of claim 4, wherein the mammal is a non-human mammal selected from the group consisting of a dog, a cat, and a horse.

7. A method of diagnosing schizophrenia or a related disorder in a subject comprising identifying a decrease in the level of palmitoylation of PSD93 when compared to a subject not diagnosed with schizophrenia or a related disorder.

8. A method of diagnosing schizophrenia or a related disorder in a subject comprising identifying a decrease in the level of palmitoylation of PSD95 when compared to a subject not diagnosed with schizophrenia or a related disorder.

9. A method for identifying an agent for treating schizophrenia or a related disorder in a subject caused by a mutation in the gene ZDHHC8, wherein the mutation decreases ZDHHC8 enzymatic activity, comprising
(i) contacting a test cell with a test agent; and
(ii) measuring the level of PSD93 palmitoylation, wherein an increase in PSD93 palmitoylation compared to a cell not contacted with a test agent bears a positive correlation with the ability of the test agent to treat schizophrenia or a related disorder.

10. A method for identifying an agent for treating schizophrenia or a related disorder in a subject caused by a mutation in the gene ZDHHC8, wherein the mutation decreases ZDHHC8 enzymatic activity, comprising
(i) contacting a test cell with a test agent; and
(ii) measuring the level of PSD95 palmitoylation, wherein an increase in PSD95 palmitoylation compared to a cell not contacted with a test agent bears a positive correlation with the ability of the test agent to treat schizophrenia or a related disorder.

11. A method for identifying an agent for treating schizophrenia or a related disorder in a subject caused by a mutation in the gene ZDHHC8, wherein the mutation decreases ZDHHC8 enzymatic activity, comprising
(i) contacting a test cell with a test agent; and
(ii) measuring the level of PSD93 membrane translocation, wherein an increase in PSD93 membrane translocation compared to a cell not contacted with a test agent bears a positive correlation with the ability of the test agent to treat schizophrenia or a related disorder.

12. A method for identifying an agent for treating schizophrenia or a related disorder in a subject caused by a mutation in the gene ZDHHC8, wherein the mutation decreases ZDHHC8 enzymatic activity, comprising
(i) contacting a test cell with a test agent; and
(ii) measuring the level of PSD95 membrane translocation, wherein an increase in PSD95 membrane translocation compared to a cell not contacted with a test agent bears a positive correlation with the ability of the test agent to treat schizophrenia or a related disorder.

13. A method for identifying an agent for treating schizophrenia or a related disorder in a subject caused by a mutation in the gene ZDHHC8, wherein the mutation decreases ZDHHC8 enzymatic activity, comprising
(i) contacting a test cell with a test agent; and
(ii) measuring the ratio of excitatory glutamatergic to inhibitory GABAergic synapses, wherein an increase in the ratio of excitatory glutamatergic to inhibitory GABAergic synapses compared to a cell not contacted with a test agent bears a positive correlations with the ability of the test agent to treat schizophrenia or a related disorder.

14. A method for identifying an agent for treating schizophrenia or a related disorder in a subject caused by a mutation in the gene ZDHHC8, wherein the mutation decreases ZDHHC8 enzymatic activity, comprising
(i) contacting a test organism with a test agent; and
(ii) measuring a behavior of the test organism, wherein an increase in the behavior compared to a test organism not contacted with the test agent bears a positive correlation with the ability of the test agent to treat schizophrenia or a related disorder.

15. The method of claim 9, 10, 11, 12, 13, or 14, wherein the test organism is a non-human transgenic animal, wherein the expression or activity of ZDHHC8 is decreased.

16. The method of claim 15, wherein the non-human transgenic animal is a mouse.

17. A method of treating a subject diagnosed as having schizophrenia or a related disorder comprising administering to the subject an effective amount of an agent wherein the agent increases the level of PSD93 palmitoylation compared to the level of PSD93 palmitoylation in a subject not administered the agent.

18. A method of treating a subject diagnosed as having schizophrenia or a related disorder comprising administering to the subject an effective amount of an agent wherein the agent increases the level of PSD95 palmitoylation compared to the level of PSD95 palmitoylation in a subject not administered the agent.

19. The method of claim 17 or 18, wherein the agent is an isolated nucleic acid encoding human ZDHHC8, operably linked to a suitable promoter.
